(19) United States
(12) Patent Application Publication
(10) Pub. No.: US 2025/0281885 A1
(43) Pub. Date: Sep. 11, 2025

(54) UREA FILTRATION DEVICE COMPRISING NANOFIBER COMPOSITIONS

(71) Applicant: ACCUDX CORPORATION, La Jolla, CA (US)

(72) Inventors: Raveendran POTTATHIL, La Jolla, CA (US); Kiran KHARAT, Aurangabad (IN)

(21) Appl. No.: 18/693,892

(22) PCT Filed: Sep. 21, 2022
(86) PCT No.: PCT/US2022/044295

§ 371 Date: Mar. 20, 2024

Related U.S. Application Data
(60) Provisional application No. 63/246,703, filed on Sep. 21, 2021.

(51) Int. Cl. B01D 69/14; A61M 1/14; B01D 61/24; B01D 71/44; B01J 23/50; B01J 23/75; B01J 23/755; B01J 23/89; B01J 31/06; B01J 31/14; B01J 31/28; B01J 35/59; B01J 37/34

(52) U.S. Cl. CPC B01D 69/145; A61M 1/155; B01D 61/243; B01D 69/14111; B01D 71/441; B01J 23/50; B01J 23/75; B01J 23/755; B01J 23/8913; B01J 23/892; B01J 31/06; B01J 31/146; B01J 31/28; B01J 35/59; B01J 37/342

(57) ABSTRACT

Provided herein are compositions, devices, and methods including nanofiber compositions comprising a polymer and nanoparticles including one or more of nickel, cobalt, silver, and tetraphenylborate, wherein the compositions are capable of binding targets in the blood related to blood disorders symptomatic of kidney disease and/or failure.

### CROSS-REFERENCE TO RELATED APPLICATIONS

This application is a U.S. National Stage Application under 35 U.S.C. § 371 of PCT Application No. PCT/US2022/044295, filed Sep. 21, 2022; which claims priority to U.S. Provisional Application No. 63/246,703, filed Sep. 21, 2021, each of which is incorporated herein by reference in its entirety.

### TECHNICAL FIELD

Compositions and methods provided herein relate to nanotechnology and medical uses thereof.

### BACKGROUND

Kidneys remove harmful particles from the blood and regulate the blood's ionic concentrations, while retaining essential ions. The harmful particles and excess water absorbed from blood are discharged from the body as urine. Under certain conditions, kidneys functions are impaired. When kidneys lose nearly 90% of their capacity of blood purification, the condition is referred to as Chronic Kidney Disease (CKD) which generally leads to “End Stage Renal Disease” (ESRD). If untreated, a patient will die due to accumulation of toxic material in the body.

The treatment for CKD/ESRD is dialysis and/or kidney transplant. Dialysis is a process of blood purification that utilizes a device such as a hemodialysis machine, which performs kidney functions outside the human body. Peritoneal dialysis is a form of hemodialysis that uses the peritoneum in a person's abdomen as the membrane to filter the blood inside the body.

Hemodialysis on average involves a patient undergoing 3 to 4 treatments per week each lasting 3 to 4 hours or longer. Despite major advances in the technology of hemodialysis and management of its complications, the morbidity and mortality of patients on dialysis remain high.

There remains a need to reduce the hemodialysis treatment time for patients in order to improve patient compliance and quality of life.

### SUMMARY

In an aspect, provided herein are nanofiber compositions including a polymer and nanoparticles including one or more of nickel, cobalt, silver, and tetraphenylborate.

In an aspect, provided herein is a cartridge including one or more membranes, where each membrane includes a nanofiber composition and where the nanofiber composition includes a polymer and one or more nanoparticles including nickel, cobalt, silver, and tetraphenylborate nanoparticles.

In an aspect, provided herein is a device including a filtration chamber configured to receive blood containing urea; and one or more membranes disposed within the filtration chamber, where each membrane includes a nanofiber composition including a polymer and nanoparticles including one or more of nickel, cobalt, silver, and tetraphenylborate, and where the nanofibers are capable of binding urea, converting urea to ammonia, and subsequently binding ammonia.

In an aspect, provided herein are methods of reducing the concentration of urea from blood, including a) providing blood containing urea to a device that includes a cartridge, where the cartridge includes one or more membranes, where each membrane includes a nanofiber composition that include a polymer and one or more of nickel, cobalt, silver, and tetraphenylborate nanoparticles; b) contacting the blood with the membrane for a sufficient amount of time to allow binding of urea and conversion of urea to ammonia; and c) pumping the blood through the cartridge at a sufficient pressure to allow binding of ammonia to the membrane, thereby reducing the concentration of urea in the blood.

In an aspect, provided herein are methods of treating a subject with a disease condition characterized by elevated blood urea concentration, the method including a) obtaining a sample of the subject's blood; b) pumping the sample through a nanofiber composition that includes a polymer and nanoparticles, where the nanoparticles include one or more of nickel, cobalt, silver, and tetraphenylborate, for a time period sufficient to allow the nanofiber composition to bind urea, convert urea to ammonia, and subsequently bind ammonia, thereby creating a filtered blood sample; and c) returning the filtered blood sample to the subject, thereby treating the subject.

In an aspect, provided herein are methods of treating a mammal for elevated blood urea concentration including a) providing blood including urea to a device that includes one or more membranes, where each membrane includes a nanofiber composition including a polymer and nanoparticles including one or more of nickel, cobalt, silver, and tetraphenylborate, and where the nanofiber composition is capable of binding urea, converting urea to ammonia, and subsequently binding ammonia; b) contacting the blood with the membrane for a sufficient amount of time to allow binding of urea and conversion to ammonia; and c) pumping the blood through the cartridge at a sufficient pressure and flow rate to allow binding of ammonia to the nanofibers, thereby reducing the concentration of urea in the blood.

In an aspect, provided herein are methods of treating a subject with a disease condition characterized by elevated urea concentration in blood, the method including: a) administering ex vivo hemofiltration to the subject where blood is removed from the subject and filtered through a device that includes a filtration chamber (1) configured to receive blood characterized by elevated urea concentration, and (2) composed of one or more membranes, where each membrane includes a nanofiber composition that includes a polymer and nanoparticles of one or more of nickel, cobalt, silver, and tetraphenylborate; b) incubating the blood with the one or more membranes for a sufficient time to allow binding of urea to the nanofiber composition, conversion of urea to ammonia, and subsequent binding of ammonia to the nanofiber composition; c) pumping the blood through the device at a pressure and flow rate sufficient to filter the blood and produce an amount of filtered blood; and d) returning the filtered blood to said subject, wherein the filtered blood is reduced in urea concentration by at least 50%.

Provided herein are uses of any of the compositions, the cartridges, and the devices described herein in the reduction of urea concentration in a sample of blood.

### BRIEF DESCRIPTION OF THE DRAWINGS

FIG. 1 is a schematic of the general layout of dialysis treatment.

FIG. 2 shows an example result of particle size analyzer demonstrating particle radii of cobalt colloidal nanoparticles to be approximately 96.6 nanometers.

FIG. 3 shows an example result of Zeta potential of analysis of silver nanoparticles.

FIG. 4 shows an example field emission scanning electron microscopy image of silver and cobalt nanofibers with sodium tetraphenylborate.

FIG. 5 shows an example field emission scanning electron microscopy image of silicon dioxide and cobalt nanofibers with sodium tetraphenylborate.

FIG. 6 shows an example field emission scanning electron microscopy image of silicon dioxide and silver nanofibers with sodium tetraphenylborate.

FIG. 7 shows an example field emission scanning electron microscopy image of copper and silver nanofibers with sodium tetraphenylborate.

FIG. 8 shows a microscopy image of blood cells exposed to the nanoparticles of silver and cobalt and tetraphenylborate material.

FIG. 9 shows an example XRD pattern obtained for nickel nanofibers in polycarbonate material.

FIG. 10 shows an example XRD pattern obtained for cobalt nanofibers in polycarbonate material.

FIG. 11 presents microscope images of blood cells. Left panel is untreated control, center panel is blood cells after dialysis through a dialyzer of the instant disclosure at a rate of 200 ml/min., right panel is blood cells after dialysis through a dialyzer of the instant disclosure at a rate of 300 ml/min.

FIG. 12A-B show an example CAD design of an example of a prototype housing for the dialyzer. FIG. 12A is a view of the inside and sides of the housing. FIG. 12B is a view of the bottom side of the housing.

### DETAILED DESCRIPTION

### Definitions

The practice of the technology will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2, or 1% to a reference quantity, level, value, concentration, measurement, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

A “nanoparticle,” as used herein, is a particle wherein the longest diameter is less than or equal to 1000 nanometers. The longest dimension of the nanoparticle may be referred to herein as the length of the nanoparticle. The shortest dimension of the nanoparticle may be referred to herein refer as the width of the nanoparticle. Nanoparticles may be composed of any appropriate material. For example, nanoparticle cores may include appropriate metals and metal oxides thereof (e.g., a metal nanoparticle core), carbon (e.g., an organic nanoparticle core) silicon and oxides thereof (e.g., a silicon nanoparticle core) or boron and oxides thereof (e.g., a boron nanoparticle core), or mixtures thereof. In embodiments, the nanoparticle has the shape of a sphere, rod, cube, triangular, hexagonal, cylinder, spherocylinder, or ellipsoid. The nanoparticle may have a diameter. For disc shaped nanoparticles, the diameter as used herein refers to the length of the disc from end to end. The impact of particle morphology is intertwined with many physiochemical parameters such as size, elasticity, surface chemistry, and bio persistence. In embodiments, the nanoparticle has a diameter of about 10 to about 1000 nanometers, about 100 to about 900 nanometers, from about 200 to about 800 nanometers, from about 300 to about 700 nanometers, or from 400 to about 600 nanometers. In some embodiments, the nanoparticle has a diameter of about 10 to about 300 nanometers. In some embodiments, the nanoparticle has a diameter of about 30 to about 150 nanometers. In some embodiments, the nanoparticle has a diameter of about 40 to about 70 nanometers.

An “inorganic nanoparticle” is used in accordance with its plain ordinary meaning and refers to a nanoparticle that does not contain carbon. For example, an inorganic nanoparticle may contain a metal or metal oxide thereof (e.g., gold nanoparticle, iron nanoparticle), silicon and oxides thereof (e.g., a silicon dioxide nanoparticle), or titanium and oxides thereof (e.g., titanium dioxide nanoparticle). In embodiments, the inorganic nanoparticle is a silicon dioxide nanoparticle. In embodiments, the inorganic nanoparticle is a metal nanoparticle. In embodiments, the nanoparticle is nickel. In embodiments, the nanoparticle is cobalt. In embodiments, the nanoparticle is silver. In embodiments, the nanoparticle is tetraphenylborate. In embodiments, the inorganic nanoparticle further includes a moiety that contains carbon.

The term “silica” is used according to its plain and ordinary meaning, is used interchangeably with “silicon dioxide” and refers to a composition (e.g., a solid composition such as a crystal, nanoparticle, or nanocrystal) containing oxides of silicon such as Si atoms (e.g., in a tetrahedral coordination) with 2 oxygen atoms surrounding a central Si atom. Nanoparticles may be composed of at least two distinct materials, one material (e.g., insoluble drug) forms the core and the other material forms the shell (e.g., silica) surrounding the core; when the shell includes Si atoms, the nanoparticle may be referred to as a silica nanoparticle. A silica nanoparticle may refer to a particle including a matrix of silicon-oxygen bonds wherein the longest diameter is typically less than or equal to 1000 nanometers.

A functionalized silica nanoparticle, as used herein, may refer to the post hoc conjugation (i.e., conjugation after the formation of the silica nanoparticle) of a moiety to the hydroxyl surface of a nanoparticle. For example, a silica nanoparticle may be further functionalized to include additional atoms (e.g., nitrogen) or chemical entities (e.g., polymeric moieties or bioconjugate group). For example, when the silica nanoparticle is further functionalized with a nitrogen containing compound, one of the surface oxygen atoms surrounding the Si atom may be replaced with a nitrogen containing moiety.

The term “polymeric” refers to a molecule including repeating subunits (e.g., polymerized monomers). For example, polymeric molecules may be based upon polyethylene glycol (PEG), poly[amino(1-oxo-1,6-hexanediyl)], poly(oxy-1,2-ethanediyloxycarbonyl-1,4-phenylenecarbonyl), tetraethylene glycol (TEG), polyvinylpyrrolidone (PVP), poly(xylene), or poly(p-xylylene).

The term “poloxamer” is used in accordance with its meaning in the art of polymer chemistry and refers to a triblock copolymer composed of a central hydrophobic block (e.g., polyoxypropylene) flanked by two hydrophilic blocks (e.g., polyoxyethylene). Poloxamers may be customized by adjusting the degree of hydrophobicity and/or hydrophilicity by extending or retracting the length of the blocks.

The term “polymerizable monomer” is used in accordance with its meaning in the art of polymer chemistry and refers to a compound that may covalently bind chemically to other monomer molecules (such as other polymerizable monomers that are the same or different) to form a polymer.

The term “branched polymer” is used in accordance with its meaning in the art of polymer chemistry and refers to a molecule including repeating subunits, wherein at least one repeating subunit (e.g., polymerizable monomer) is covalently bound to an additional subunit substituent (e.g., resulting from a reaction with a polymerizable monomer). For example, a branched polymer has the formula:

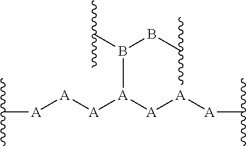

wherein ‘A’ is the first repeating subunit and ‘B’ is the second repeating subunit. In embodiments, the first repeating subunit (e.g., polyethylene glycol) is optionally different than the second repeating subunit (e.g., polymethylene glycol).

The term “block copolymer” is used in accordance with its ordinary meaning and refers to two or more portions (e.g., blocks) of polymerized monomers linked by a covalent bond. In embodiments, a block copolymer is a repeating pattern of polymers. In embodiments, the block copolymer includes two or more monomers in a periodic (e.g., repeating pattern) sequence. For example, a diblock copolymer has the formula: -B-B-B-B-B-B-A-A-A-A-A-, where ‘B’ is a first subunit and ‘A’ is a second subunit covalently bound together. A triblock copolymer therefore is a copolymer with three distinct blocks, two of which may be the same (e.g., -A-A-A-A-A-B-B-B-B-B-B-A-A-A-A-A-) or all three are different (e.g., -A-A-A-A-A-B-B-B-B-B-B-C-C-C-C-C-) where ‘A’ is a first subunit, ‘B’ is a second subunit, and ‘C’ is a third subunit, covalently bound together.

As used herein, the term “electrospinning” is used in accordance with its plain ordinary meaning and refers to a fiber production method which uses electric force to draw charged threads of polymer solutions or polymer melts up to fiber diameters in the order of some hundred nanometers. Electrospinning shares characteristics of both electro spraying and conventional solution dry spinning of fibers. The process does not require the use of coagulation chemistry or high temperatures to produce solid threads from solution. This makes the process particularly suited to the production of fibers using large and complex molecules. Electrospinning from molten precursors is also practiced; this method ensures that no solvent can be carried over into the final product.

As used herein, the term “nanofiber” is used in accordance with its plain ordinary meaning and refers to fibers with diameters in the nanometer range. Nanofibers can be generated from different polymers and hence have different physical properties and application potentials. Examples of natural polymers include collagen, cellulose, silk fibroin, keratin, gelatin and polysaccharides such as chitosan and alginate. Examples of synthetic polymers include poly(lactic acid) (PLA), polycaprolactone (PCL), polyurethane (PU), poly(lactic-co-glycolic acid) (PLGA), poly(3-hydroxybutyrate-co-3-hydroxyvalerate) (PHBV), and poly(ethylene-co-vinylacetate) (PEVA). Polymer chains are connected via covalent bonds. The diameters of nanofibers depend on the type of polymer used and the method of production. All polymer nanofibers are unique for their large surface area-to-volume ratio, high porosity, appreciable mechanical strength, and flexibility in functionalization compared to their microfiber counterparts. There exist many different methods to make nanofibers, including drawing, electrospinning, self-assembly, template synthesis, and thermal-induced phase separation. Electrospinning is the most commonly used method to generate nanofibers because of the straightforward setup, the ability to mass-produce continuous nanofibers from various polymers, and the capability to generate ultrathin fibers with controllable diameters, compositions, and orientations.

In embodiments, the nanofiber compositions include a polymer that provides stability. In embodiments, the nanofiber compositions include a polymer that provides stability, where the polymer is one or more of silicon dioxide, polyurethane prepolymer (PUP), polylactic acid (PLA), polycarbonate, polyvinyl alcohol (PVA), polyacrylic acid (PAA), polyethylene glycol (PEG), and polyvinyl pyrrolidone (PVP). In embodiments, the nanofiber compositions include a polymer that is capable of binding ammonia. In embodiments, the nanofiber compositions include a polymer that is capable of binding ammonia where the polymer is a silicon dioxide polymer.

In embodiments described herein, provided are nanofibers composed of one or more of nanoparticles capable of binding urea and/or converting urea to ammonia. In embodiments described herein, provided are nanofibers composed of one or more of nanoparticles capable of binding urea and/or converting urea to ammonia, where the one or more nanoparticles are nickel nanoparticles, cobalt nanoparticles, silver nanoparticles, and/or tetraphenylborate nanoparticles.

As used herein, the term “membrane” is used in accordance with its plain ordinary meaning and refers to a selective barrier; it allows some things to pass through but stops others. Such things may be molecules, ions, or other small particles. Biological membranes include cell membranes (outer coverings of cells or organelles that allow passage of certain constituents); nuclear membranes, which cover a cell nucleus; and tissue membranes, such as mucosae and serosae. Synthetic membranes are made by humans for use in laboratories and industry (such as chemical plants). In embodiments described herein, membranes include a composite of nanofibers composed of polymers and nanoparticles.

As used herein, the term “cartridge” refers to a configuration or housing that may encase nanofibers or membranes as described herein.

As used herein, the term “target” is used in accordance with its plain ordinary meaning and refers to a cell or molecule or region of interest which is captured by any one or more of a nanoparticle, polymer, nanofiber, composition, and combinations thereof described herein. In embodiments, a target is a molecule. In embodiments, a target is a compound. In embodiments, a target is urea. In embodiments, a target is ammonia.

As used herein, the terms “disease” or “condition” are used in accordance with their plain ordinary meaning and refer to a state of being or health status of a patient or subject capable of being treated with the compounds or methods provided herein. The disease may be a kidney disease. The disease may be a blood disease. The disease may be a condition characterized by elevated concentration of a compound in the blood. The disease may be a condition characterized by elevated concentration of urea in the blood.

As used herein, the term “blood disorder” or “blood disease” is used in accordance with its plain ordinary meaning and refers to a condition that affects one or more parts of the blood and prevent blood from doing its job. They can be acute or chronic. Many blood disorders are inherited. Other causes include other diseases, side effects of medicines, and a lack of certain nutrients in your diet. In embodiments, blood disorder refers to elevated urea concentration.

As used herein, the term “dialysis” is used in accordance with its plain ordinary meaning and refers to a treatment that filters and purifies the blood using a machine. The kidneys filter blood by removing waste and excess fluid from the body. This waste is sent to the bladder to be eliminated from the body as urine. Dialysis performs the function of the kidneys if they are failing or have failed. This helps keep fluids and electrolytes in balance when the kidneys can't do their job. There are three different types of dialysis: hemodialysis, peritoneal dialysis, and continuous renal replacement therapy.

As used herein, the term “hemodialysis” is used in accordance with its plain ordinary meaning and refers to process uses an artificial kidney (hemodialyzer) to remove waste and extra fluid from the blood. The blood is removed from the body and filtered through the artificial kidney. The filtered blood is then returned to the body with the help of a dialysis machine. To get the blood to flow to the artificial kidney, a doctor will perform surgery to create an entrance point (vascular access) into your blood vessels. The three types of entrance points are: 1) Arteriovenous (AV) fistula, which connects an artery and a vein; 2) AV graft, which is a looped tube; 3) Vascular access catheter, which may be inserted into the large vein in the neck. Both the AV fistula and AV graft are designed for long-term dialysis treatments. People who receive AV fistulas are healed and ready to begin hemodialysis two to three months after their surgery. People who receive AV grafts are ready in two to three weeks. Catheters are designed for short-term or temporary use. Hemodialysis treatments usually last three to five hours and are performed about three times per week.

As used herein, the term “peritoneal dialysis” is used in accordance with its plain ordinary meaning and refers to dialysis involving surgery to implant a peritoneal dialysis (PD) catheter into the abdomen. The catheter helps filter blood through the peritoneum, a membrane in the abdomen. During treatment, a special fluid called dialysate flows into the peritoneum. The dialysate absorbs waste. Once the dialysate draws waste out of the bloodstream, it is drained from the abdomen. This process takes a few hours and needs to be repeated four to six times per day. However, the exchange of fluids can be performed while the subject is sleeping or awake.

As used herein, the terms “continuous renal replacement therapy” or “hemofiltration” are used in accordance with its plain ordinary meaning and refer to therapy used primarily in the intensive care unit for people with acute kidney failure. A machine passes the blood through tubing. A filter then removes waste products and water. The blood is returned to the body, along with replacement fluid. This procedure is performed 12 to 24 hours a day, generally every day.

As used herein the term “filtration” is used in accordance with its plain ordinary meaning and refers to a physical or chemical separation process that separates solid matter and fluid from a mixture using a filter medium. Biological filtration may take place inside an organism, or the biological component may be grown on a medium in the material being filtered. Removal of solids, emulsified components, organic chemicals and ions may be achieved by ingestion and digestion, adsorption or absorption. Inside mammals, reptile and birds, the kidneys function by renal filtration in which the glomerulus selectively removes undesirable constituents such as urea, followed by selective reabsorption of many substances essential for the body to maintain homeostasis. The complete process is termed excretion.

As used herein, the term “urea” also known as “carbamide”, is an organic compound with chemical formula CO(NH₂)₂. This amide has two —NH₂ groups joined by a carbonyl (C═O) functional group. Urea serves an important role in the metabolism of nitrogen-containing compounds by animals and is the main nitrogen-containing substance in the urine of mammals. It is a colorless, odorless solid, highly soluble in water, and practically non-toxic (LD₅₀ is 15 g/kg for rats). Dissolved in water, it is neither acidic nor alkaline. The body uses it in many processes, most notably nitrogen excretion. The liver forms it by combining two ammonia molecules (NH₃) with a carbon dioxide (CO₂) molecule in the urea cycle.

As used herein, the term “potassium” refers to a is a mineral and an electrolyte. It helps muscles work, including the muscles that control heartbeat and breathing. The body uses the potassium it needs. The extra potassium that the body does not need is removed from the blood by kidneys. If a subject has kidney disease, the kidneys cannot remove extra potassium in the right way, and too much potassium can stay in the blood. Having too much potassium, hyperkalemia, in the blood can be dangerous as it may lead to heart attack.

As used herein, the term “inhibitor” is used in accordance with its plain ordinary meaning and refers to a compound (e.g., compounds described herein) that reduces activity when compared to a control, such as absence of the compound or a compound with known inactivity.

As used herein, the terms “inhibition”, “inhibit”, “inhibiting” and the like in reference to an interaction that negatively affects (e.g., decreasing or inactivates or kills) the activity or function of a target. Inhibition can mean a decrease in functional or live target by 10%, 20%, 30%, 40%, 50%, 60%, 70%, 80%, 90% or more in comparison to a control in the absence of the inhibitor. In certain instances, expression or activity is 1.5-fold, 2-fold, 3-fold, 4-fold, 5-fold, 10-fold or lower than the expression or activity in the absence of the antagonist.

As used herein, the term “contacting” refers to allowing two species to react, interact, or physically touch, where the two species may be a nanoparticle, nanofiber, nanofiber composition, or polymer as described herein and a cell, protein, antibody, aptamer, or another compound.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

As used herein, the term “prevent” refers to a decrease in the occurrence of disease symptoms in a patient. The prevention may be complete (no detectable symptoms) or partial, such that fewer symptoms are observed than would likely occur absent treatment. As used herein, prevent refers to reduction of targets (e.g., urea) such that a patient is prevented from experiencing the detrimental effects of elevated blood urea concentration.

As used herein, the term “patient” or “subject in need thereof” refers to a living organism suffering from or prone to a disease or condition that can be treated by methods or compositions provided herein. Non-limiting examples include humans, other mammals, bovines, rats, mice, dogs, monkeys, goat, sheep, cows, deer, and other non-mammalian animals. In some embodiments, a patient is human. In some embodiments, a patient is canine. In some embodiments, a patient is feline.

As used herein, the term “effective amount” is an amount sufficient for a composition as described herein to accomplish a stated purpose relative to the absence of the composition (e.g., achieve the effect for which it is administered, prevent infection, reduce target activity, and the like). An example of an “effective amount” is an amount sufficient to contribute to the prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of symptoms associated with elevated blood urea concentration or the like. In embodiments, the effective amount refers to a number of nanofibers, nanoparticles, and/or membranes comprising nanofibers as described herein to affect a reduction in blood urea concentration, and/or bind ammonia. The exact amounts will depend on the purpose of the treatment, and will be ascertainable by one skilled in the art using known techniques.

For any composition described herein, the therapeutically effective amount can be initially determined from assays. Target and filter composite concentration that are capable of achieving the methods described herein, as measured using the methods described herein or known in the art.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “compound”, is used in accordance with its plain ordinary meaning and refers to a substance formed when two or more chemical elements are chemically bonded together. As described herein, a compound may be a target compound. In embodiments, the target compound is urea. In embodiments, the target compound is ammonia.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the rate of infection in the absence of a filter as described herein (including embodiments and examples).

As used herein, the terms “specific”, “specifically”, “specificity”, or the like of a composition refers to the composition's ability to cause a particular action, such as inhibition, to a particular molecular target with minimal or no action to other proteins in the cell.).

As used herein, the term “solution” refers to a liquid mixture in which the minor component (e.g., a solute or compound) is uniformly distributed within the major component (e.g., a solvent). In embodiments, the solution includes nanoparticles.

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

As used herein, the terms “bind” and “bound” are used in accordance with its plain and ordinary meaning and refer to the association between atoms or molecules. The association can be direct or indirect. For example, bound atoms or molecules may be direct, e.g., by covalent bond or linker (e.g., a first linker or second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waal's bond(s)/interactions, hydrogen bond(s), polar bond(s), or combinations or mixtures thereof).

### Composition

In an aspect, provided herein are nanofiber compositions including a polymer and nanoparticles including one or more of nickel, cobalt, silver, and tetraphenylborate.

In embodiments, nanofiber compositions provided herein include a polymer, where the polymer includes one or more of silicon dioxide, polyurethane prepolymer (PUP), polylactic acid (PLA), polycarbonate, polyvinyl alcohol (PVA), polyacrylic acid (PAA), polyethylene glycol (PEG), and polyvinyl pyrrolidone (PVP). In embodiments, the polymer is non-electroconductive. In embodiments, the polymer is silicon dioxide. In embodiments, the polymer is polyurethane prepolymer (PUP). In embodiments, the polymer is polylactic acid (PLA). In embodiments, the polymer is polycarbonate. In embodiments, the polymer is polyvinyl alcohol (PVA). In embodiments, the polymer is polyacrylic acid (PAA). In embodiments, the polymer is polyethylene glycol (PEG). In embodiments, the polymer is polyvinyl pyrrolidone (PVP). In embodiments, nanofiber compositions provided herein include a polymer including a combination of silicon dioxide, polyurethane prepolymer (PUP), polylactic acid (PLA), polycarbonate, polyvinyl alcohol (PVA), polyacrylic acid (PAA), polyethylene glycol (PEG), and polyvinyl pyrrolidone (PVP). In embodiments, nanofiber compositions provided herein include a polymer including a combination of silicon dioxide and polyvinyl pyrrolidone (PVP).

In embodiments, nanofiber compositions provided herein include a polymer and nanoparticles, where the nanoparticles are composed on one or more of nickel, cobalt, silver, and tetraphenylborate. In embodiments, the nanofiber compositions include nickel nanoparticles. In embodiments, the nanofiber compositions include cobalt nanoparticles. In embodiments, the nanofiber compositions include silver nanoparticles. In embodiments, the nanofiber compositions include tetraphenylborate nanoparticles. In embodiments, nanofiber compositions provided herein include a polymer and nanoparticles, where the nanoparticles are composed on a combination of nickel, cobalt, silver, or tetraphenylborate. In embodiments, nanofiber compositions provided herein include a polymer and silver and nickel nanoparticles. In embodiments, nanofiber compositions provided herein include a polymer and silver, nickel, and tetraphenylborate nanoparticles. In embodiments, nanofiber compositions provided herein include a polymer and silver and cobalt nanoparticles. In embodiments, nanofiber compositions provided herein include a polymer and silver, cobalt, and tetraphenylborate nanoparticles. The nanoparticles may be spun to form a nanofiber. The nanoparticles may be spun with other materials to form a nanofiber.

In embodiments, nanofiber compositions provided herein include a polymer composed of one or more of silicon dioxide, PVP, PUP, PLA, polycarbonate, PVA, PAA, and PEG, and nanoparticles of one or more of nickel, silver, cobalt, and tetraphenylborate. In embodiments, the nanofiber compositions include a polymer including silicon dioxide and further including nickel nanoparticles. In embodiments, the nanofiber compositions include a polymer comprising silicon dioxide and further include cobalt nanoparticles. In embodiments, the nanofiber compositions include a polymer comprising silicon dioxide and further include silver nanoparticles. In embodiments, the nanofiber compositions include a polymer comprising silicon dioxide and further include tetraphenylborate nanoparticles. In embodiments, the nanofiber compositions include a polymer comprising silicon dioxide and further include nickel and cobalt nanoparticles. In embodiments, the nanofiber compositions include a polymer comprising silicon dioxide and further include nickel and silver nanoparticles. In embodiments, the nanofiber compositions include a polymer comprising silicon dioxide and further include cobalt and silver nanoparticles. In embodiments, the nanofiber compositions include a polymer comprising silicon dioxide and further include nickel, cobalt and tetraphenylborate nanoparticles. In embodiments, the nanofiber compositions include a polymer comprising silicon dioxide and further include nickel, silver, and tetraphenylborate nanoparticles. In embodiments, the nanofiber compositions include a polymer including silicon dioxide and further include cobalt, silver, and tetraphenylborate nanoparticles.

In embodiments, the nanofiber compositions include a polymer including polyvinyl pyrrolidone (PVP) and further include nickel nanoparticles. In embodiments, the nanofiber compositions include a polymer including polyvinyl pyrrolidone (PVP) and further include cobalt nanoparticles. In embodiments, the nanofiber compositions include a polymer including polyvinyl pyrrolidone (PVP) and further include silver nanoparticles. In embodiments, the nanofiber compositions include a polymer including polyvinyl pyrrolidone (PVP) and further include tetraphenylborate nanoparticles. In embodiments, the nanofiber compositions include a polymer including polyvinyl pyrrolidone (PVP) and further include nickel and cobalt nanoparticles. In embodiments, the nanofiber compositions include a polymer including polyvinyl pyrrolidone (PVP) and further include nickel and silver nanoparticles. In embodiments, the nanofiber compositions include a polymer including polyvinyl pyrrolidone (PVP) and further include cobalt and silver nanoparticles. In embodiments, the nanofiber compositions include a polymer including polyvinyl pyrrolidone (PVP) and further include nickel, cobalt and tetraphenylborate nanoparticles. In embodiments, the nanofiber compositions include a polymer including polyvinyl pyrrolidone (PVP) and further include nickel, silver, and tetraphenylborate nanoparticles. In embodiments, the nanofiber compositions include a polymer comprising polyvinyl pyrrolidone (PVP) and further include cobalt, silver, and tetraphenylborate nanoparticles.

In embodiments, the nanofiber compositions include a polymer including polyurethane prepolymer (PUP), and further include nickel nanoparticles. In embodiments, the nanofiber compositions include a polymer including polyurethane prepolymer (PUP), and further include cobalt nanoparticles. In embodiments, the nanofiber compositions include a polymer including polyurethane prepolymer (PUP), and further include silver nanoparticles. In embodiments, the nanofiber compositions include a polymer including polyurethane prepolymer (PUP), and further include tetraphenylborate nanoparticles. In embodiments, the nanofiber compositions include a polymer including polyurethane prepolymer (PUP), and further include nickel and cobalt nanoparticles. In embodiments, the nanofiber compositions include a polymer including polyurethane prepolymer (PUP), and further include nickel and silver nanoparticles. In embodiments, the nanofiber compositions include a polymer including polyurethane prepolymer (PUP), and further include cobalt and silver nanoparticles. In embodiments, the nanofiber compositions include a polymer including polyurethane prepolymer (PUP), and further include nickel, cobalt and tetraphenylborate nanoparticles. In embodiments, the nanofiber compositions include a polymer including polyurethane prepolymer (PUP), and further include nickel, silver, and tetraphenylborate nanoparticles. In embodiments, the nanofiber compositions include a polymer including polyurethane prepolymer (PUP), and further include cobalt, silver, and tetraphenylborate nanoparticles.

In embodiments, the nanofiber compositions include a polymer including polylactic acid (PLA) and further include nickel nanoparticles. In embodiments, the nanofiber compositions include a polymer including polylactic acid (PLA) and further include cobalt nanoparticles. In embodiments, the nanofiber compositions include a polymer including polylactic acid (PLA) and further include silver nanoparticles. In embodiments, the nanofiber compositions include a polymer including polylactic acid (PLA) and further include tetraphenylborate nanoparticles. In embodiments, the nanofiber compositions include a polymer including polylactic acid (PLA) and further include nickel and cobalt nanoparticles. In embodiments, the nanofiber compositions include a polymer including polylactic acid (PLA) and further include nickel and silver nanoparticles. In embodiments, the nanofiber compositions include a polymer including polylactic acid (PLA) and further include cobalt and silver nanoparticles. In embodiments, the nanofiber compositions include a polymer including polylactic acid (PLA) and further include nickel, cobalt and tetraphenylborate nanoparticles. In embodiments, the nanofiber compositions include a polymer including polylactic acid (PLA) and further include nickel, silver, and tetraphenylborate nanoparticles. In embodiments, the nanofiber compositions include a polymer including polylactic acid (PLA) and further include cobalt, silver, and tetraphenylborate nanoparticles.

In embodiments, the nanofiber compositions include a polymer including polycarbonate and further include nickel nanoparticles. In embodiments, the nanofiber compositions include a polymer including polycarbonate and further include cobalt nanoparticles. In embodiments, the nanofiber compositions include a polymer including polycarbonate and further include silver nanoparticles. In embodiments, the nanofiber compositions include a polymer including polycarbonate and further include tetraphenylborate nanoparticles. In embodiments, the nanofiber compositions include a polymer including polycarbonate and further include nickel and cobalt nanoparticles. In embodiments, the nanofiber compositions include a polymer including polycarbonate and further include nickel and silver nanoparticles. In embodiments, the nanofiber compositions include a polymer including polycarbonate and further include cobalt and silver nanoparticles. In embodiments, the nanofiber compositions include a polymer including polycarbonate and further include nickel, cobalt and tetraphenylborate nanoparticles. In embodiments, the nanofiber compositions include a polymer including polycarbonate and further include nickel, silver, and tetraphenylborate nanoparticles. In embodiments, the nanofiber compositions include a polymer including polycarbonate and further include cobalt, silver, and tetraphenylborate nanoparticles.

In embodiments, the nanofiber compositions include a polymer including polyvinyl alcohol (PVA) and further include nickel nanoparticles. In embodiments, the nanofiber compositions include a polymer including polyvinyl alcohol (PVA) and further include cobalt nanoparticles. In embodiments, the nanofiber compositions include a polymer including polyvinyl alcohol (PVA) and further include silver nanoparticles. In embodiments, the nanofiber compositions include a polymer including polyvinyl alcohol (PVA) and further include tetraphenylborate nanoparticles. In embodiments, the nanofiber compositions include a polymer including polyvinyl alcohol (PVA) and further include nickel and cobalt nanoparticles. In embodiments, the nanofiber compositions include a polymer including polyvinyl alcohol (PVA) and further include nickel and silver nanoparticles. In embodiments, the nanofiber compositions include a polymer including polyvinyl alcohol (PVA) and further include cobalt and silver nanoparticles. In embodiments, the nanofiber compositions include a polymer including polyvinyl alcohol (PVA) and further include nickel, cobalt and tetraphenylborate nanoparticles. In embodiments, the nanofiber compositions include a polymer including polyvinyl alcohol (PVA) and further include nickel, silver, and tetraphenylborate nanoparticles. In embodiments, the nanofiber compositions include a polymer including polyvinyl alcohol (PVA) and further include cobalt, silver, and tetraphenylborate nanoparticles.

In embodiments, the nanofiber compositions include a polymer including polyacrylic acid (PAA) and further include nickel nanoparticles. In embodiments, the nanofiber compositions include a polymer including polyacrylic acid (PAA) and further include cobalt nanoparticles. In embodiments, the nanofiber compositions include a polymer including polyacrylic acid (PAA) and further include silver nanoparticles. In embodiments, the nanofiber compositions include a polymer including polyacrylic acid (PAA) and further include tetraphenylborate nanoparticles. In embodiments, the nanofiber compositions include a polymer including polyacrylic acid (PAA) and further include nickel and cobalt nanoparticles. In embodiments, the nanofiber compositions include a polymer including polyacrylic acid (PAA) and further include nickel and silver nanoparticles. In embodiments, the nanofiber compositions include a polymer including polyacrylic acid (PAA) and further include cobalt and silver nanoparticles. In embodiments, the nanofiber compositions include a polymer including polyacrylic acid (PAA) and further include nickel, cobalt and tetraphenylborate nanoparticles. In embodiments, the nanofiber compositions include a polymer including polyacrylic acid (PAA) and further include nickel, silver, and tetraphenylborate nanoparticles. In embodiments, the nanofiber compositions include a polymer including polyacrylic acid (PAA) and further include cobalt, silver, and tetraphenylborate nanoparticles.

In embodiments, the nanofiber compositions include a polymer including polyethylene glycol (PEG) and further include nickel nanoparticles. In embodiments, the nanofiber compositions include a polymer including polyethylene glycol (PEG) and further include cobalt nanoparticles. In embodiments, the nanofiber compositions include a polymer including polyethylene glycol (PEG) and further include silver nanoparticles. In embodiments, the nanofiber compositions include a polymer including polyethylene glycol (PEG)) and further include tetraphenylborate nanoparticles. In embodiments, the nanofiber compositions include a polymer including polyethylene glycol (PEG) and further include nickel and cobalt nanoparticles. In embodiments, the nanofiber compositions include a polymer including polyethylene glycol (PEG) and further include nickel and silver nanoparticles. In embodiments, the nanofiber compositions include a polymer including polyethylene glycol (PEG) and further include cobalt and silver nanoparticles. In embodiments, the nanofiber compositions include a polymer including polyethylene glycol (PEG) and further include nickel, cobalt and tetraphenylborate nanoparticles. In embodiments, the nanofiber compositions include a polymer including polyethylene glycol (PEG) and further include nickel, silver, and tetraphenylborate nanoparticles. In embodiments, the nanofiber compositions include a polymer including polyethylene glycol (PEG) and further include cobalt, silver, and tetraphenylborate nanoparticles.

In embodiments, the nanofiber compositions described herein are capable of binding urea. In embodiments, the nanofiber compositions described herein are capable of converting urea to ammonia. In embodiments, the nanofiber compositions described herein are capable of binding ammonia. In embodiments, the nanofiber compositions described herein are capable of binding urea, converting urea to ammonia, and binding ammonia.

In embodiments, provided herein are nanofiber compositions composed of nanoparticles, where the nanoparticle has a diameter of about 5 to about 1000 nanometers. In embodiments, the nanoparticles have an average diameter of about 10 to about 1000 nanometers, about 100 to about 900 nanometers, from about 200 to about 800 nanometers, from about 300 to about 700 nanometers, or from 400 to about 600 nanometers. In some embodiments, the nanoparticle has a diameter of about 10 to about 500, about 20 to about 400, about 30 to about 300, about 40 to about 200, or about 50 to about 100 nanometers. In some embodiments, the nanoparticles have an average diameter of about 10 to about 250, about 20 to about 200, about 30 to about 150, or about 40 to about 100 nanometers. In some embodiments, the nanoparticles have an average diameter of about 10 nanometers, about 20 nanometers, about 30 nanometers, about 40 nanometers, about 50 nanometers, about 60 nanometers, about 70 nanometers, about 80 nanometers, about 90 nanometers, about 100 nanometers, about 110 nanometers, about 120 nanometers, about 130 nanometers, about 140 nanometers, about 150 nanometers, about 160 nanometers, about 170 nanometers, about 180 nanometers, or about 190 nanometers. In some embodiments, the nanoparticles have a diameter of about 200 nanometers, about 300 nanometers, about 400 nanometers, about 500 nanometers, about 600 nanometers, about 700 nanometers, about 800 nanometers, about 900 nanometers, or about 1000 nanometers. In some embodiments, the nanoparticle has a diameter of about 10 to about 300. In some embodiments, nanoparticles have an average diameter of about 20 to about 150 nanometers. In some embodiments, nanoparticles have an average diameter of about 40 to about 70 nanometers. In some embodiments, the nanoparticle has a diameter of about 10 nanometers. In some embodiments, the nanoparticles have an average diameter of about 20 nanometers. In some embodiments, the nanoparticles have an average diameter of about 30 nanometers. In some embodiments, nanoparticles have an average diameter of about 40 nanometers. In some embodiments, the nanoparticles have an average diameter of about 50 nanometers. In some embodiments, nanoparticles have an average diameter of about 60 nanometers. In some embodiments, the nanoparticle has a diameter of about 70 nanometers. In some embodiments, nanoparticles have an average diameter of about 80 nanometers. In some embodiments, the nanoparticle has a diameter of about 90 nanometers. In some embodiments, nanoparticles have an average diameter of about 100 nanometers. In some embodiments, nanoparticles have an average diameter of about 110 nanometers. In some embodiments, nanoparticles have an average diameter of about 120 nanometers. In some embodiments, the nanoparticle has a diameter of about 130 nanometers. In some embodiments, nanoparticles have an average diameter of about 140 nanometers. In some embodiments, nanoparticles have an average diameter of about 150 nanometers. Nanoparticle diameter may be any value or subrange within the recited ranges, including endpoints.

In embodiments, provided herein are nanofiber compositions where the polymer and nanoparticles are interwoven together by electrospinning to form fibers.

### Nanofiber Manufacture

In an aspect, provided herein are nanofiber compositions produced by electrospinning.

### Membranes

In an aspect, provided herein is a membrane composed of nanofiber compositions as described herein. The membranes may be arranged to form a filter or composite of membranes. The filter or membrane composite may be housed in a cartridge or arranged in a device such as a dialyzer to be used in a hemodialysis machine or a peritoneal dialysis machine.

In embodiments, provided herein is a membrane including nanofibers, where the nanofibers include a polymer and one or more of nickel nanoparticles, cobalt nanoparticles, silver nanoparticles, and tetraphenylborate nanoparticles. In embodiments, the membranes include nanofibers including a polymer, where the polymer includes one or more of silicon dioxide, polyvinyl pyrrolidone (PVP), polyurethane prepolymer (PUP), polylactic acid (PLA), polycarbonate, polyvinyl alcohol (PVA), polyacrylic acid (PAA), and polyethylene glycol (PEG). In embodiments, the polymer is non-electroconductive. In embodiments, the polymer is silicon dioxide. In embodiments, the polymer is polyvinyl pyrrolidone (PVP). In embodiments, the polymer is polyurethane prepolymer (PUP). In embodiments, the polymer is polylactic acid (PLA). In embodiments, the polymer is polycarbonate. In embodiments, the polymer is polyvinyl alcohol (PVA). In embodiments, the polymer is polyacrylic acid (PAA). In embodiments, the polymer is polyethylene glycol (PEG). In embodiments, the polymer is silicon dioxide and polyvinyl pyrrolidone (PVP).

In embodiments, provided herein are membranes that include nanofibers as described herein. Specifically, the nanofibers include a polymer and nanoparticles, where the nanoparticles are composed of one or more of nickel, cobalt, silver, and tetraphenylborate. In embodiments, the nanoparticles are nickel. In embodiments, the nanoparticles are cobalt. In embodiments, the nanoparticles are silver. In embodiments, the nanoparticles are tetraphenylborate. In embodiments, the nanoparticles are nickel and silver. In embodiments, the nanoparticles are cobalt and silver. In embodiments, the nanoparticles are nickel and cobalt. In embodiments, the nanoparticles are nickel, silver, and tetraphenylborate. In embodiments, the nanoparticles are cobalt, silver, and tetraphenylborate. In embodiments, the nanoparticles are nickel, cobalt, and tetraphenylborate. The nanoparticles may be spun to form a nanofiber. The nanoparticles may be spun with other materials to form a nanofiber.

In embodiments, provided herein are membranes including nanofibers including a polymer including SiO₂, and nanoparticles of silver and cobalt nanoparticles and further includes tetraphenylborate nanoparticles. In embodiments, provided herein are membranes including nanofibers including a polymer including PVP, and nanoparticles of silver and cobalt nanoparticles and further includes tetraphenylborate nanoparticles. In embodiments, provided herein are membranes including nanofibers including a polymer including SiO₂, and nanoparticles of silver, and nickel nanoparticles and further includes tetraphenylborate nanoparticles. In embodiments, provided herein are membranes including nanofibers including a polymer including PVP, and nanoparticles of silver and nickel nanoparticles and further includes tetraphenylborate nanoparticles.

In embodiments, provided herein are membranes composed of nanofibers, where the nanofibers are capable of binding urea, converting urea to ammonia, and/or binding ammonia. In embodiments, the nanofibers are capable of binding urea and converting urea to ammonia. In embodiments, the nanofibers composed of one or more of nickel, cobalt, and silver nanoparticles are capable of binding urea and converting urea to ammonia. In embodiments, the nanofibers are capable of binding ammonia. In embodiments, the nanofibers composed of one or more of silicon dioxide and tetraphenylborate are capable of binding ammonia.

### Devices

In an aspect, provided herein is a cartridge including one or more membranes, where each membrane includes a nanofiber composition and where the nanofiber composition includes a polymer and one or more nanoparticles including nickel, cobalt, silver, and tetraphenylborate nanoparticles.

In embodiments, membranes of the cartridges provided herein include nanofibers including a polymer, where the polymer includes one or more of silicon dioxide, polyurethane prepolymer (PUP), polylactic acid (PLA), polycarbonate, polyvinyl alcohol (PVA), polyacrylic acid (PAA), PEG, and PVP. In embodiments, the polymer is non-electroconductive. In embodiments, the polymer is silicon dioxide. In embodiments, the polymer is polyurethane prepolymer (PUP). In embodiments, the polymer is polylactic acid (PLA). In embodiments, the polymer is polycarbonate. In embodiments, the polymer is polyvinyl alcohol (PVA). In embodiments, the polymer is polyacrylic acid (PAA). In embodiments, the polymer is polyethylene glycol (PEG). In embodiments, the polymer is polyvinyl pyrrolidone (PVP). In embodiments, membranes of the cartridges provided herein include nanofibers including a polymer, where the polymer includes a combination of silicon dioxide, polyurethane prepolymer (PUP), polylactic acid (PLA), polycarbonate, polyvinyl alcohol (PVA), polyacrylic acid (PAA), polyethylene glycol (PEG), and, polyvinyl pyrrolidone (PVP). In embodiments, membranes of the cartridges provided herein include nanofibers including a polymer, where the polymer includes a combination of silicon dioxide and, polyvinyl pyrrolidone (PVP).

In embodiments, membranes of the cartridges provided herein include nanofibers including a polymer and nanoparticles, where the nanoparticles are composed on one or more of nickel, cobalt, silver, and tetraphenylborate. In embodiments, the nanoparticles are nickel. In embodiments, the nanoparticles are cobalt. In embodiments, the nanoparticles are silver. In embodiments, the nanoparticles are tetraphenylborate. The nanoparticles may be spun to form a nanofiber. The nanoparticles may be spun with other materials to form a nanofiber.

In embodiments, provided herein are cartridges composed of membranes including nanofibers, where the nanofibers include a polymer of silicon dioxide (SiO₂) and nanoparticles of nickel and silver. In embodiments, provided herein are cartridges composed of membranes including nanofibers, where the nanofibers include a polymer of silicon dioxide (SiO₂) and nanoparticles of silver and cobalt. In embodiments, provided herein are cartridges composed of membranes further including and tetraphenylborate-nanoparticles.

In embodiments, provided herein are cartridges including one or more membranes composed of nanofibers as described herein, where the nanofibers are capable of binding urea, converting urea to ammonia, and binding ammonia.

In an aspect, provided herein is a device including a filtration chamber configured to receive blood containing urea; and one or more membranes disposed within the filtration chamber, wherein each membrane includes a nanofiber composition including a polymer and nanoparticles including one or more of nickel, cobalt, silver, and tetraphenylborate, and where the nanofibers are capable of binding urea, converting urea to ammonia, and subsequently binding ammonia.

In embodiments, the membranes disposed within the device include the nanofiber compositions as described herein.

In embodiments, the device provided herein is wearable.

In embodiments, the device provided herein is formatted for ex vivo filtration.

### Methods of Use

In an aspect, provided herein are methods of reducing the concentration of urea from blood, including a) providing blood containing urea to a device that includes a cartridge, where the cartridge includes one or more membranes, where each membrane includes a nanofiber composition that includes a polymer including one or more of silicon dioxide, polyvinyl pyrrolidone (PVP), polyurethane prepolymer (PUP), polylactic acid (PLA), polycarbonate, polyvinyl alcohol (PVA), polyacrylic acid (PAA), and polyethylene glycol (PEG) and one or more of nickel, cobalt, silver, and tetraphenylborate nanoparticles; b) contacting the blood with the membrane for a sufficient amount of time to allow binding of urea and conversion of urea to ammonia; and c) pumping the blood through the cartridge at a sufficient pressure to allow binding of ammonia to the membrane, thereby reducing the concentration of urea in the blood.

In an aspect, provided herein are methods of treating a subject with a disease condition characterized by elevated blood urea concentration, the method including a) obtaining a sample of the subject's blood; b) pumping the sample through a nanofiber composition that includes a polymer and nanoparticles, where the nanoparticles include one or more of nickel, cobalt, silver, and tetraphenylborate, for a time period sufficient to allow the nanofiber composition to bind urea, convert urea to ammonia, and subsequently bind ammonia, thereby creating a filtered blood sample; and c) returning the filtered blood sample to the subject, thereby treating the subject.

In an aspect, provided herein are methods of treating a mammal for elevated blood urea concentration including a) providing blood including urea to a device that includes one or more membranes, where each membrane includes a nanofiber composition including a polymer including one or more of silicon dioxide, polyvinyl pyrrolidone (PVP), polyurethane prepolymer (PUP), polylactic acid (PLA), polycarbonate, polyvinyl alcohol (PVA), polyacrylic acid (PAA), and polyethylene glycol (PEG) and nanoparticles including one or more of nickel, cobalt, silver, and tetraphenylborate, and where the nanofiber composition is capable of binding urea, converting urea to ammonia, and subsequently binding ammonia; b) contacting the blood with the membrane for a sufficient amount of time to allow binding of urea and conversion to ammonia; and c) pumping the blood through the cartridge at a sufficient flowrate to allow binding of ammonia to the nanofibers, thereby reducing the concentration of urea in the blood.

In an aspect, provided herein are methods of treating a subject with a disease condition characterized by elevated urea concentration in blood, the method including: a) administering ex vivo hemofiltration to the subject where blood is removed from the subject and filtered through a device that includes a filtration chamber (1) configured to receive blood characterized by elevated urea concentration, and (2) composed of one or more membranes, where each membrane includes a nanofiber composition that includes a polymer including one or more of silicon dioxide, polyvinyl pyrrolidone (PVP), polyurethane prepolymer (PUP), polylactic acid (PLA), polycarbonate, polyvinyl alcohol (PVA), polyacrylic acid (PAA), and polyethylene glycol (PEG) and nanoparticles of one or more of nickel, cobalt, silver, and tetraphenylborate; b) incubating the blood with the one or more membranes for a sufficient time to allow binding of urea to the nanofiber composition, conversion of urea to ammonia, and subsequent binding of ammonia to the nanofiber composition; c) pumping the blood through the device at a flow rate sufficient to filter the blood and produce an amount of filtered blood; and d) returning filtered blood to said subject, wherein the filtered blood is reduced in urea concentration by at least 50%.

In embodiments, provided herein are methods including providing blood, where providing blood is achieved by pumping of blood from a subject into any one of the various device embodiments described herein.

Pumping of blood may be achieved using standard medical grade pumps.

In embodiments, provided herein are methods of contacting a blood sample with any one of the various membranes described herein, for a time period sufficient for binding of urea and conversion of urea to ammonia. In embodiments, a sufficient time is about 5 minutes to about 2 hours. In embodiments, a sufficient time is 10 minutes. In embodiments, a sufficient time is 15 minutes. In embodiments, a sufficient time is 20 minutes. In embodiments, a sufficient time is 25 minutes. In embodiments, a sufficient time is 30 minutes. In embodiments, a sufficient time is 35 minutes. In embodiments, a sufficient time is 40 minutes. In embodiments, a sufficient time is 45 minutes. In embodiments, a sufficient time is 50 minutes. In embodiments, a sufficient time is 55 minutes. In embodiments, a sufficient time is 60 minutes. In embodiments, a sufficient time is 65 minutes. In embodiments, a sufficient time is 70 minutes. In embodiments, a sufficient time is 75 minutes. In embodiments, a sufficient time is 80 minutes. In embodiments, a sufficient time is 85 minutes. In embodiments, a sufficient time is 90 minutes. In embodiments, a sufficient time is 95 minutes. In embodiments, a sufficient time is 100 minutes. In embodiments, a sufficient time is 105 minutes. In embodiments, a sufficient time is 110 minutes. In embodiments, a sufficient time is 115 minutes. In embodiments, a sufficient time is 120 minutes.

In embodiments, provided herein are methods of pumping a blood sample through a cartridge as described herein at a sufficient flowrate to allow binding of ammonia to the membrane, thereby reducing the concentration of urea in the blood. In embodiments, a sufficient flow rate is about 25 milliters of blood per minute (ml/min) to about 400 milliters of blood per minute (ml/min). In embodiments, a sufficient flow rate is about 50 ml/min. In embodiments, a sufficient flow rate is about 100 ml/min. In embodiments, a sufficient flow rate is about 150 ml/min. In embodiments, a sufficient flow rate is about 200 ml/min. In embodiments, a sufficient flow rate is about 250 ml/min. In embodiments, a sufficient flow rate is about 300 ml/min. In embodiments, a sufficient flow rate is about 350 ml/min. In embodiments, a sufficient flow rate is about 400 ml/min.

In embodiments, provided herein are methods of treating a subject with a disease condition characterized by elevated blood urea concentration, where the disease is kidney failure, chronic kidney disease (CKD), acute kidney injury (AKI), or End Stage Renal Disease (ESRD). In embodiments, the disease is kidney disease. In embodiments, the disease is chronic kidney disease (CKD). In embodiments, the disease is acute kidney injury (AKI). In embodiment, the disease is End Stage Renal Disease (ESRD).

In embodiments, provided herein are methods of treating a subject that involve pumping a sample of the subject's blood through a nanofiber composition that includes a polymer and nanoparticles, where the nanoparticles include one or more of nickel, cobalt, silver, and tetraphenylborate, for a time period sufficient to allow the nanofiber composition to bind urea, convert urea to ammonia, and subsequently bind ammonia, thereby creating a filtered blood sample. In embodiments, pumping may be achieved using standard medical grade pumps known in the art. In embodiments, a time period sufficient to allow the nanofiber composition to bind urea, convert urea to ammonia, and subsequently bind ammonia, thereby creating a filtered blood sample is about 5 minutes to about 2 hours.

In embodiments, the methods of use of the device include utilizing a device including any of the nanofiber compositions described and waiting a sufficient amount of time for the composition to bind a target. In embodiments, the target is one or more of urea and ammonia. In embodiments, a sufficient amount of time includes from about 1 minute to about 1 hour. In embodiments, a sufficient amount of time includes 1, 2, 3, 4, 5, 6, 7, 8, 9, 10 or greater than 10 minutes. In embodiments, a sufficient amount of time includes 10, 20, 30, 40, 50, or about 60 minutes. In embodiments, a sufficient amount of time includes from about 1 hour to about 24 hours. In embodiments, a sufficient amount of time includes about 1, 2, 3, 4, 5, 6, 7, 8, 9, 10, 11, 12, or greater than 12 hours. In embodiments, a sufficient amount of time includes from about 12 to about 24 hours. In embodiments, a sufficient amount of time includes about 12, 13, 14, 15, 16, 17, 18, 19, 20, 21, 22, 23, 24, or greater than 24 hours.

In embodiments, provided herein are methods of treating a subject that involve returning a filtered blood sample to the subject, thereby treating the subject. In embodiments, returning filtered blood involves pumping filtered blood back into a subject using standard medical grade pumps known in the art.

Provided herein are uses of any of the compositions, the cartridges, and the devices described herein in the reduction of urea concentration in a sample of blood.

### EXAMPLES

### Example 1. Study Design

Despite major advances in the technology of hemodialysis and management of its complications, the morbidity and mortality of patients on dialysis remain high (Kidney Int Rep. 2020 November; 5(11): 1856-1869.) Also, post-dialysis recovery time can be twice as long as the treatment time, during which patients report feeling ill, lethargic, and depressed, preventing most patients from holding a full-time job. More importantly, life expectancies for end-stage renal disease patients have improved little in the past two decades, with almost no change (<1 year increase) for those 50 years of age and older. The objective of the experiments described herein is to improve removal of urea from the blood and to reduce hemodialysis treatment time for patients to less than half of the current time. These aims improve both health outcomes and quality of life for those on hemodialysis.

Blood contains particles of many different sizes and types, including cells, proteins, dissolved ions, and organic waste products. Some of these particles, such as proteins like hemoglobin, are essential for the body. Others, such as urea, creatinine, potassium, phosphorus, and excess water must be removed from the blood or be maintained within a certain range, otherwise these accumulate and interfere with normal metabolic processes. (https://www.urmc.rochester.edu/encyclopedia/content.aspx?ContentTypeID=90&ContentID=P02316)

Experiments designed herein had the objective of providing a cartridge containing nanofiber compositions which include polymers and multivalent nanoconjugates of nanoparticles of one or more of nickel, silver, cobalt, and tetraphenylborate for the removal of urea from the blood by first binding the urea in blood, hydrolyzing urea (that is the conversion of urea to ammonia) and subsequent binding of ammonia, thereby producing blood reduced in urea concentration.

The nanoparticle composition for blood urea reduction when used instead of existing dialysis machines reduce dialysis duration from four (4) to less than two (2) hours by reducing urea removal time. An added benefit is nearly doubling the number of patients being treated without additional capital costs. Thus, there is the potential to reduce treatment costs to the patient (and/or insurance reimbursement) and improve return on investment (ROI) for the service provider. Most importantly, patient compliance and quality of life improve as the treatment time is reduced.

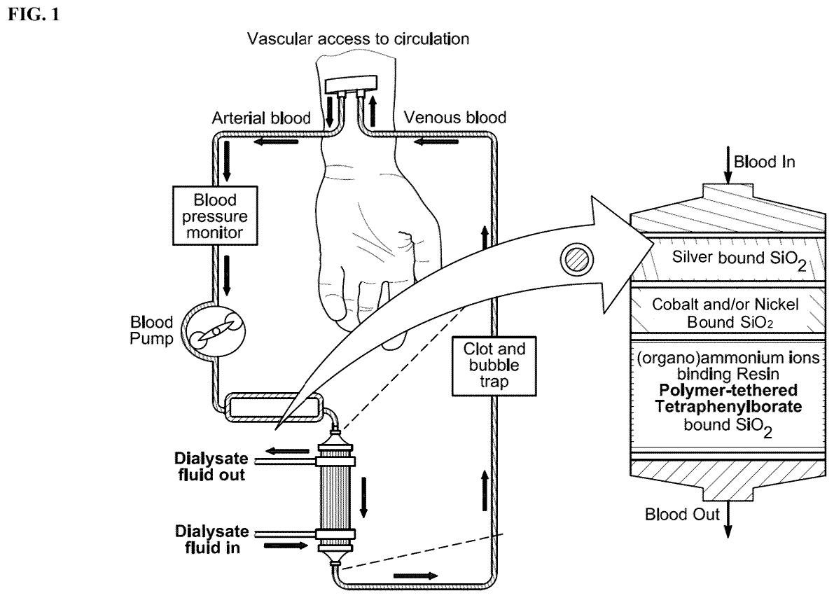

As described in the schematic in FIG. 1 about the procedure, blood enters first a urea filtration cartridge. Urea is converted into ammonia within the nanofiber compositions housed in the cartridge. The blood is then absorbed in situ by nanofibers as described herein. The blood flows through the cartridge. The cartridge includes a nanofiber composition includes a polymer and optionally mutually exclusive layers of one or more of nickel, silver, cobalt, and tetraphenylborate nanoparticles. In an embodiment, where the nanofiber composition includes polymers of silicon dioxide and polymers of PVP and includes silver and cobalt nanoparticles, the silver or cobalt react with urea in the blood. The urea is degraded into ammonia and CO₂. Silicon dioxide and tetraphenylborate absorb free ammonia generated during the degradation of urea. The blood flows through the cartridge several times for about 5 minutes to about 120 min at about 100 ml/min to about 300 ml per min.

The material comprised of nanofibers as described herein binds urea and converts it to ammonia, which is then captured by the tetraphenylborate material. Then, the blood that has been reduced in urea concentration is obtained and directed back to the subject.

This nanofiber-based cartridge for blood urea reduction has been shown to efficiently remove urea from blood and reduce treatment duration from 4 hours to less than 1 hour.

The rationale to design the nanofiber cartridge was as follows: a) minimal to no possible damage to blood cells: the minimum size of blood passage through the cartridge should be such that blood cells are not damaged during treatment; b) blood flow rate should be well matched with hemodialysis such that blood flow rate maximum and minimum through the cartridge does not lead to clotting or cell damage; c) structural stability: nanoparticles used in the cartridge manufacturing should not get dislodged and be carried into the subject with treated blood; d) surface area offered should ensure maximum possible contact of active material with the flowing blood to maximize waste elimination while minimizing the treatment time.

At the same time, a cartridge was required to have uniform inter-fiber space and uniform external profile of the fiber. Furthermore, it also required mechanical strength. The challenge was that high speed winding of fiber gives aligned fibers that are likely to give uniform inter-fiber space but would have poor mechanical strength and slow speed winding of fiber gives non-aligned fibers (random) that have better mechanical strength but may not give uniform inter-fiber space.

Dog and rabbit testing was conducted to test for safety and efficacy. The first trial was on a dog and the second trial was on a rabbit.

### Example 2: Nanoparticle Testing

The approach for the artificial kidney was to create cartridges comprising nanostructures of metals such as nickel, cobalt silver, or combinations thereof in a matrix capable of catalyzing the conversion of urea into ammonia. Additional layers of tetraphenylborate which is known to quantitatively absorb ammonia were used as well. After screening a number of metal nanoconjugates, it was determined that nanofibers of silver and cobalt as well as silver and nickel were the most efficient matrices for the conversion of urea into ammonia. A surprising finding was that silicon dioxide polymers meant to provide stability to the nanofiber was also capable of binding ammonia.

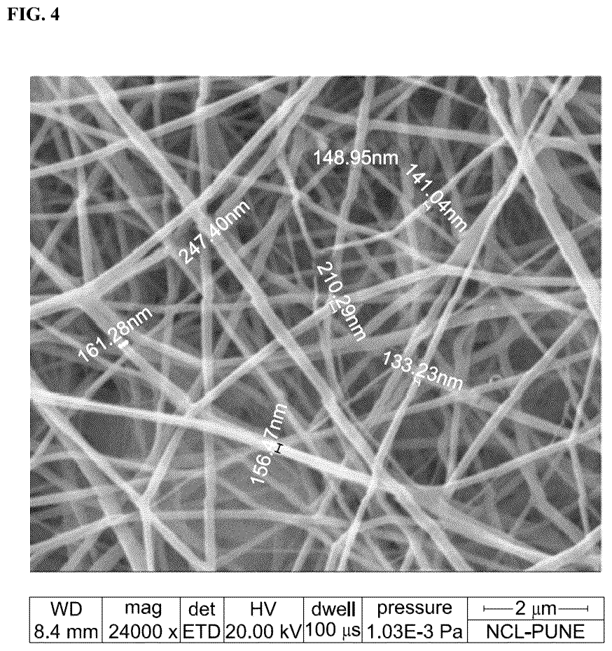

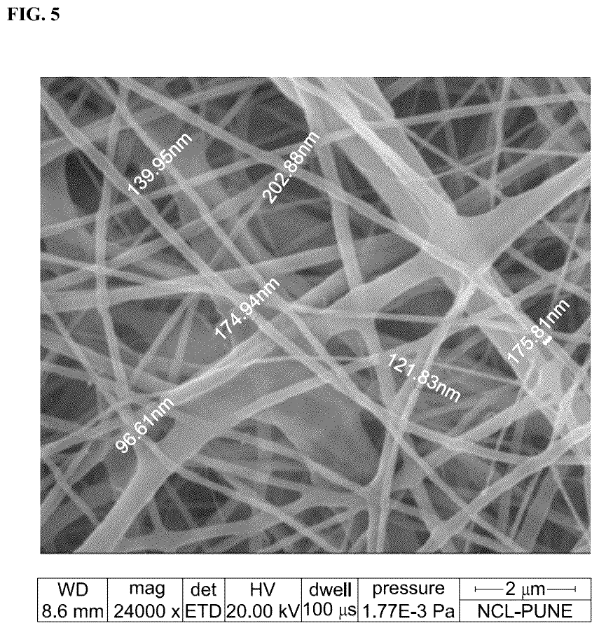

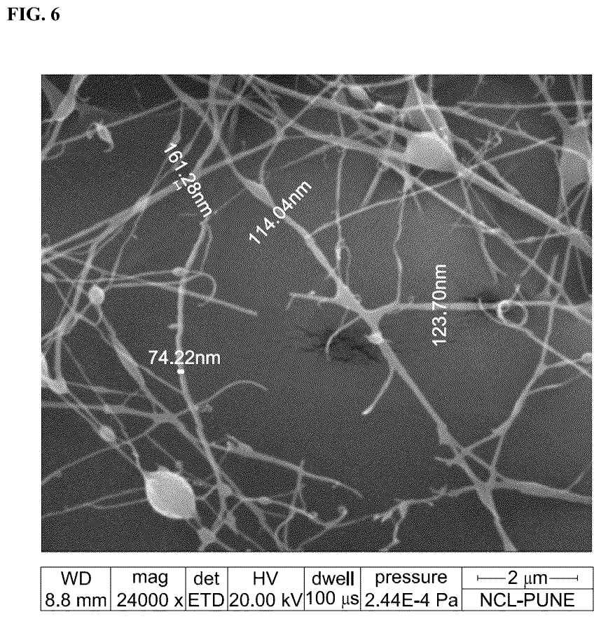

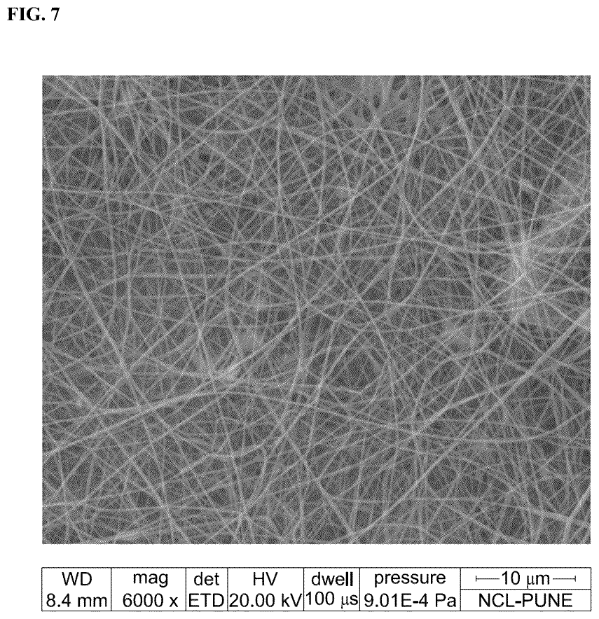

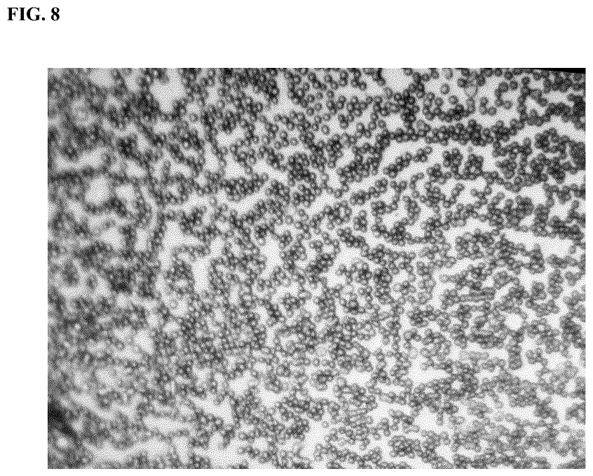

Experiments were undertaken to develop and validate a commercially scalable process for creating nano-silver and nano-cobalt fibers that can be incorporated into 3-D printed cartridges. The structures were characterized by Fourier transform infrared spectroscopy (FT-IR). The nanofiber conjugates were analyzed using field emission electron microscopy (FIGS. 4-7). The matrices were shown to effectively remove urea from the blood both in vitro and in vivo (see Tables 1-4 below). Introduction of copper-silver nanoconjugates in the cartridges effectively removed creatinine. The stability of the cartridge matrices was analyzed for leaching and thermal stability. The matrices were also tested for cellular toxicity. The newly synthesized nanofibers were used for toxicity study. These nanofibers were exposed to blood for three hours and then observed under microscope. The cells were found to be healthy and normal after 1 hour (FIG. 8) and up to three hours (data not shown).

Effectiveness of the material was tested using animals with kidney disease. Limited studies done in dogs and rabbits showed effective removal of urea within one hour without any deleterious effects on haemopoietic cells and clinical chemistry.

Described herein are experiments supporting proof of concept for a nanofiber-based cartridge to reduce blood urea concentration and to reduce treatment duration from 4 hours to estimated 1 hour.

### Silver Nanoparticles Synthesis

A desired amount of silver nitrate was dissolved in absolute ethanol ([Ag2+]=0.056 M, 0.111 M and 0.333 M) (Solution A). Another mixture was obtained by mixing potassium hydroxide and hydrazine monohydrate (molar ratio for N2H4/Ag2+=2.5, 5 and 10) together (Solution B). Then, solution “A” was poured into solution “B” immediately with vigorous, continued magnetic stirring at room temperature. The overall reaction time was about 2 hours. The resultant product was washed thoroughly with deionized water for removal of reaction residues followed by washing with acetone. Finally, the black particles were soaked in acetone in a closed bottle for further characterization.

### Cobalt and Silver Nanoparticles

To the previous colloidal solution of silver particles, 1.5 mmol of CO₂ (CO)₈ (530 mg, 0.5 equivalents,) was added. The solution kept at 120° C. for 10 minutes, after which the temperature was increased to 180° C. for 1 hour. Then, the solution was kept at room temperature and the nanoparticles (NP) were collected with a magnet. NPs were washed four times by re-dispersion in hexane (5 mL) and addition of 50 mL of isopropanol.

### Nanoparticle Analysis

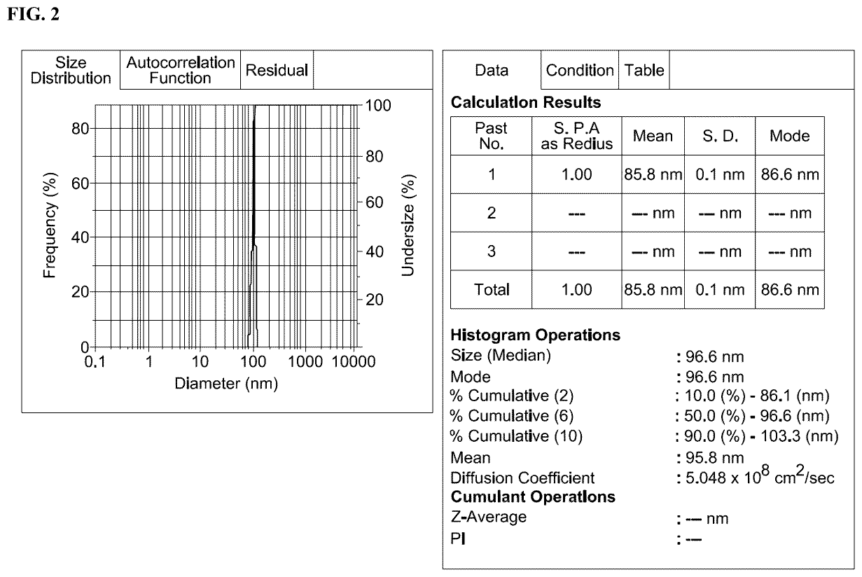

Particle size analysis of cobalt nanoparticles: The nanoparticles were analyzed using particles size analyzer. The average particle size was determined to be 96.6 nm (see for example FIG. 2).

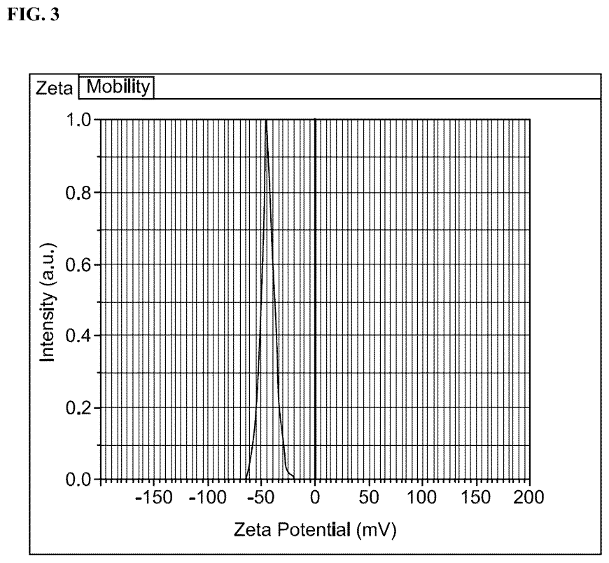

Silver Nanoparticles: Zeta potential of silver nanoparticles were found −43.8 mV (FIG. 3).

### Example 3: Estimation of Urea from Blood

### Effect of Silver on Blood Urea

Blood samples were collected from a pathology lab for the experiments. The BUN (Blood Urea Nitrogen) value was estimated from blood in presence of silver nanoparticles. Blood was distributed into 6 different test tubes (1 ml in each test tube) containing 0, 0.25, 0.5, 1, 1.5 and 2 μg/mL of silver nanoparticles (Table 1). After 2 minutes of incubation at room temperature, the BUN value was measured by using biochemical analyzer (Table 1). Data shows that after addition of 1 μg/mL silver nanoparticles, 1±0.5 mMol/L urea was degraded from blood.

TABLE 1
Estimation of urea from blood in presence of silver nanoparticles.

| | | Silver | BUN value after |
|---|---|---|---|
| Tube | Blood | nanoparticles | Silver addition |
| number | (ml) | μg/mL | mMol/L |
| 1 | 1 ml | 0.00 | 4.5 ± 0.5 |
| 2 | 1 ml | 0.25 | 4.0 ± 0.5 |
| 3 | 1 ml | 0.50 | 2.61 ± 0.5 |
| 4 | 1 ml | 1.00 | 0.39 ± 0.05 |
| 5 | 1 ml | 1.50 | 0.21 ± 0.05 |
| 6 | 1 ml | 2.00 | 0.19 ± 0.05 |

### Effect of Cobalt on Blood Urea

Blood samples obtained from a pathology lab were used for the experiment. The BUN (Blood Urea Nitrogen) value was estimated from blood containing cobalt nanoparticles. Blood was distributed into 6 different test tubes (1 ml each) containing 0, 0.25, 0.5, 1, 1.5 and 2 μg/mL-1 of cobalt nanoparticles. After 2 minutes of incubation at room temperature, the BUN value was measured by using biochemical analyzer (Table 2). Data showed that, 0.5 μg/mL cobalt nanoparticles degraded urea up to 0.4±0.02 mMol/L.

TABLE 2
Estimation of urea from Blood in presence of cobalt nanoparticles.

| | | Cobalt | BUN value after |
|---|---|---|---|
| Tube | Blood | nanoparticles | Cobalt addition |
| number | (ml) | μg/mL | mMol/L |
| 1 | 1 ml | 0.00 | 5.9 ± 0.5 |
| 2 | 1 ml | 0.25 | 2.6 ± 0.5 |
| 3 | 1 ml | 0.50 | 0.4 ± 0.02 |
| 4 | 1 ml | 1.00 | 0.28 ± 0.05 |
| 5 | 1 ml | 1.50 | 0.16 ± 0.05 |
| 6 | 1 ml | 2.00 | 0.08 ± 0.05 |

### Silicon Dioxide Polymer for the Absorption of Ammonia

Ammonia was estimated by Nessler's reagent method in presence of the silicon dioxide nanoparticles (surrogate for SiO₂ polymer). For estimation of ammonia, a stock solution of (NH₃—N₅ ml/100 ml dH₂O (50 mg/L)) was prepared. In four test tubes, 5 ml (5 mg/L) of ammonia stock solution were added. In these tubes, 0, 10, 25, 50 μg/mL of silicon dioxide nanoparticles were added. After 5 minutes of incubation at room temperature, in each test tube, 1 ml of KNa Tartarate (filtered before use) and 1 ml of Nesslers reagent was added. The tubes were kept for 5 minutes at room temperature and the optical density was measured at 425 nm (Table 3). The data showed that the silicon dioxide polymer itself binds ammonia.

TABLE 3
Estimation of ammonia in presence of Silicon
dioxide (crude) nanoparticles.

| | Stock solution of | Silicon dioxide | |
|---|---|---|---|
| | standard | polymer | Ammonia |
| Tubes | NH₃—N | μg/mL | estimation |
| 1 | 5 ml | 0 | 4.8 mg/L |
| 2 | 5 ml | 10 | 3.9 mg/L |
| 3 | 5 ml | 25 | 2.4 mg/L |
| 4 | 5 ml | 50 | 1.7 mg/L |

### Estimation of Ammonia in Presence of Na-teraphenylborate.

Na-Tetraphenylborate resins have been used for ammonia reduction (Cameron et al., 2002). Experiments were conducted to test whether nanoparticles of sodium tetraphenylborate would be suitable for removing ammonia produced from reduction of urea. Ammonia was estimated by Nessler's reagent method in presence of the Na-tetraphenylborate. The ammonia solution of 5 mg/L was used for the assay. Data showed in presence of the 8 μg/mL Na-tetraphenylborate, the ammonia was estimated at about 1.30 mg/L (Table 4) and thus absorbed from the starting amount. The conclusion from this data was that the sodium tetraphenylborate nanoparticles bind ammonia and provided a significant reduction in ammonia concentration.

TABLE 4
Estimation of ammonia in presence of Na-tetraphenyl borate.

| | Stock solution of | Na-tetraphenylborate | Ammonia |
|---|---|---|---|
| Tubes | standard NH₃—N | μg/mL | estimation |
| 1 | 5 ml | 0 | 5.02 mg/L |
| 2 | 5 ml | 2 | 4.13 mg/L |
| 3 | 5 ml | 4 | 2.89 mg/L |
| 4. | 5 ml | 8 | 1.30 mg/L |
| 5. | 5 ml | 16 | 0.52 mg/L |

### Example 3: Nanofiber Preparation

### Nanofibers Including Silver, Cobalt, Tetraphenylborate, and PVP

Using silver nitrate (AgNO₃) (Sigma-Aldrich), cobalt nitrate (Co(NO₃)₂) (Sigma-Aldrich) and a mixture of stabilizer polyvinylpyrrolidone (PVP) (0.01% w/v ratio) with 1 mM sodium tetraphenylborate (TPB) solution as main chemicals, solutions were prepared with varying salt to polymer ratios. A typical solution was prepared by mixing the specific amount of AgNO₃ and Co(NO₃)₂ in 1 ml of water via magnetic stirring for half an hour and then 1 ml of acetic acid was added followed by magnetic stirring for another half an hour. Acetic acid was added to avoid the hydrolysis of PVA. Five (5) grams of 15 wt. % (percentage by weight) aqueous PVA solution was then added, and the solution was left for vigorous magnetic stirring until a viscous and uniform solution was formed. The obtained solution was transferred to a plastic syringe with a gauge 20 (internal diameter=0.603 mm) stainless steel needle at its end. An ESPIN Nano (V2) (India) was used for electrospinning. The nozzle-collector distance was kept constant at 10 cm and the voltage was 22 kV while keeping the solution flow-rate constant at 0.5 ml/hour. The electrospinning was carried out at room temperature and a relative humidity of nearly 69% was recorded during the process. The fibers were collected on microscopic aluminum foil for specific characterization. The samples were left overnight in a furnace at 80° C. to remove the moisture followed by calcination in a furnace at 475° C. for 2 hours under ambient conditions. The heating rate was 5° C./min and once the calcination cycle was over, the furnace was allowed to cool down to room temperature before removing the samples.

### Nanofibers Including Cobalt, Tetraphenylborate, and PVP and SiO₂

In atypical procedure, 4.80 grams ethanol and 6.87 grams acetic acid were mixed as the base solution, followed by adding a mixture of stabilizer PVP (0.01% w/v ratio) with 1 mM sodium tetraphenylborate (TPB) solution to adjust the viscosity. The mixture was stirred at 30° C. for 6 hours to ensure the dissolution of PVP. Co(NO₃)₂ and TEOS (tetraethyl orthosilicate; Purity: 98%; Sigma Aldrich) was then added into the solution and stirred for 1 hour to get Co(NO₃)₂/PVP precursor solution for electrospinning. The Co(NO₃)₂/PVP precursor solution was placed in a needle with a steel tip with a constant feeding rate of 0.2 mm/minute. The needle was connected to a high-voltage power supply and positioned horizontally on a clamp, with a piece of flat aluminum foil placed 15 cm from the tip of the needle to collect the nanofibers. After applying a voltage of 20 kV, the precursor solution droplet at the tip became highly electrified and the induced charges were evenly distributed on its surface. As a result, the droplets were stretched into thread form under both electrostatic repulsions between the surface charges and Coulombic force exerted by the electric field. At the same time, the diameter of the fiber was reduced from micrometer to nanometer due to the evaporation of the solvent. Then, the nanofiber was attracted to the collector in a non-woven mat form.

### Nanofibers Including Silver, Tetraphenylborate, PVP, and SiO₂

The major substances including TEOS (tetraethyl orthosilicate; Purity: 98%; Sigma Aldrich) and silver nitrates (Sigma-Aldrich), in addition to a mixture of stabilizer PVP (0.01% w/v ratio) with 1 mM Sodium tetraphenylborate (TPB) solution (polyvinylpyrrolidone; PVPK25, MW=1300000; Purity: 98%) and butanol (Solubility: 77 g/L; Purity: 99.9%; Merck) were used for preparing the dope solution.

Dope solution with concentration of 0.1 g (PVP)/mL (TEOS+Ag NO₃+butanol) was prepared: initially, 14 ml of butanol and 24 ml of TEOS were mixed and well-stirred at 80° C. for 30 minutes. Then, 4 grams of PVP was added to this mixture and mixing was then continued at 120° C. for 90 min. The resultant solution was then kept for 24 hours under ambient conditions for relaxing the polymer chains. Viscosity and conductivity of solution were evaluated for obtaining the right rheology characteristic for electro spinning process.

### Nanofibers Including Nickel, Tetraphenylborate, PVP, and SiO₂

The major substances including TEOS (tetraethyl orthosilicate; Purity: 98%; Sigma Aldrich) and nickel 11 acetate (NiAc) (Sigma-Aldrich), in addition to a mixture of stabilizer PVP (0.01% w/v ratio) with 1 mM Sodium tetraphenylborate (TPB)solution (polyvinylpyrrolidone; PVPK25, MW=1300000; Purity: 98%;) and butanol (Solubility: 77 g/lit; Purity: 99.9%; Merck) were used for preparing the dope solution.

Dope solution with concentration of 0.1 g (PVP)/mL (TEOS+NiAc+butanol) was prepared: initially, 14 ml of butanol and 24 ml of TEOS+Nickel TT acetate were mixed and well-stirred at 80° C. for 30 min.

Then, 4 grams of PVP was added to this mixture and the mixing was continued at 120° C. for 90 min.

The resultant solution was then kept for 24 h under ambient conditions for relaxing the polymer chains. Viscosity and conductivity of solution were evaluated for obtaining the right rheology characteristic for electro spinning process.

### Example 4: Nanofiber Stability

Elemental analysis was undertaken to test for possible leaching of metal ions in blood or water samples.

In search of the stability of the nanoconjugates, microwave plasma atomic emission spectrometry (MP-AES) analysis was performed. For sample preparation, the nanofibers were kept in a water at pH 5 for 24 hours. After 24 hours, the water samples were given for the MP-AES analysis. This was repeated at pH 7.2 and pH 8.8. Experimental set up is shown in Tables 5A (silver) and 5B (Nickel).

Results shown in Table 6A (Silver) and 6B (Nickel) demonstrate that there was no leaching of metal ions was found in samples pH 5, pH 7.2 and at pH 8.8. See Table 6 below.

TABLE 5A
Nanofibers stability in water at different pH

| Sample. no | Sample code | Conc. And Content |
|---|---|---|
| 1 | Negative control | 1 ppm AgNo3 |
| 2 | Positive control | BLANK FILM OF |
| | | POLYCARBONATE |
| | | WITHOUT DRUG |
| 3 | TEST 1 | IN FILM, AgNO₃ 10%, AT pH 5 |
| | | after 24 hrs |
| 4 | TEST 2 | IN FILM, AgNO₃ 10%, AT pH 7.2 |
| | | after 24 hrs |
| 5 | TEST 3 | IN FILM, AgNO₃ 10%, AT pH 8.8 |
| | | after 24 hrs |

TABLE 5B
Nanofibers stability in water at different pH

| | Sample | |
|---|---|---|
| Sr. no | code | Conc.\Content |
| 1 | −ve C | 1 ppm NiO |
| 2 | +ve C | BLANK FILM OF POLYCARBONATE |
| | | WITHOUT DRUG |
| 3 | TEST 1 | IN FILM, NiO 10%, AT pH 5 |
| | | after 24 hrs |
| 4 | TEST 2 | IN FILM, NiO 10%, AT pH 7.2 |
| | | after 24 hrs |
| 5 | TEST 3 | IN FILM, NiO 10%, AT pH 8.8 |
| | | after 24 hrs |

TABLE 6A
Stability of Silver at different pH analyzed by MP-AES.

| | Sample | | Ag leached |
|---|---|---|---|
| Sample. no | code | Conc.\Content | ppm |
| 1 | Negative | 1 ppm | 0.27 |
| | Control | | |
| 2 | Positive | BLANK | 0.00 |
| | Control | | |
| 3 | TEST 1 | AT pH 5 after 24 | 0.07 |
| | | hrs | |
| 4 | TEST 2 | AT pH 7.2 after 24 | 0.00 |
| | | hrs | |
| 5 | TEST 3 | AT pH 8.8 after 24 | 0.00 |
| | | hrs | |

TABLE 6B
Stability of Nanofibers with Nickel Silver
at different pH analyzed by MP-AES.

| | Sample | | Ni leached |
|---|---|---|---|
| Sample. no | code | Conc.\Content | ppm |
| 1 | −ve C | 1 ppm Ni | 0.27 |
| 2 | +ve C | BLANK | 0.00 |
| 3 | TEST 1 | AT pH 5 after 24 | 0.07 |
| | | hrs | |
| 4 | TEST 2 | AT pH 7.2 after 24 | 0.00 |
| | | hrs | |
| 5 | TEST 3 | AT pH 8.8 after 24 | 0.00 |
| | | hrs | |

In search of the duration of the stability of the nanoconjugates as well as the stability in blood, microwave plasma atomic emission spectrometry (MP-AES) analysis was performed. For sample preparation, silver or cobalt nanofibers were kept in a blood at pH 7 for 12 or 24 hours. After 12 or 24 hours, the water samples were given for the MP-AES analysis. Experimental set up is shown in Tables 7A (silver) and 7B (Cobalt).

TABLE 7A
Silver Nanofiber stability at different duration

| Sr. no | Sample code | Conc. And Content |
|---|---|---|
| 1 | A1 | 1 ppm AgNO₃ |
| 2 | B | BLANK FILM OF |
| | | POLYMER WITHOUT DRUG |
| 3 | C | IN FILM, AgNO₃ 10%, AT pH 7 |
| | | after 12 hrs |
| 4 | D | IN FILM, AgNO₃ 10%, AT pH 7 |
| | | after 24 hrs |

TABLE 7B
Cobalt Nanofibers stability at different duration

| Sr. no | Sample code | Conc. And Content |
|---|---|---|
| 1 | A2 | 1 ppm Cobalt Chloride |
| 2 | B | BLANK FILM OF |
| | | POLYMER WITHOUT DRUG |
| 3 | C | IN FILM, Cobalt Chloride, |
| | | 10%, AT pH 7 |
| | | after 12 hrs |
| 4 | D | IN FILM, Cobalt Chloride, |
| | | 10%, AT pH 7 |
| | | after 24 hrs |

In further studies, the nanofibers of nickel and nanofibers of cobalt were independently kept in blood of pH 7 for 12 and 24 hours. After that the blood samples were given for the MP-AES analysis. Results showed no leaching of metal into the sample was observed (example data shown in Table 8). This data demonstrated the stability of the nanofiber for duration (12 and 24 hours) as well as to exposure to blood.

TABLE 8A
Nickel Nanofiber stability in blood

| | Sample | | Ni leached |
|---|---|---|---|
| Sample. no | code | Conc.\Content | ppm |
| 1 | −ve C | 1 ppm Ni | 1.57 |
| 2 | +ve C | BLANK | 0.00 |
| 3 | TEST 1 | AT pH 7 after 24 | 0.00 |
| | | hrs | |
| 4 | TEST 2 | AT pH 7 after 24 | 0.00 |
| | | hrs | |
| 5 | TEST 3 | AT pH 7 after 24 | 0.00 |
| | | hrs | |

TABLE 8B
Cobalt Nanofiber Stability in Blood

| | Sample | | Co leached |
|---|---|---|---|
| Sample. no | code | Conc.\Content | ppm |
| 1 | −ve C | 1 ppm Co | .92 |
| 2 | +ve C | BLANK | 0.00 |
| 3 | TEST 1 | AT pH 7 after 24 | 0.00 |
| | | hrs | |
| 4 | TEST 2 | AT pH 7 after 24 | 0.00 |
| | | hrs | |
| 5 | TEST 3 | AT pH 7 after 24 | 0.00 |
| | | hrs | |

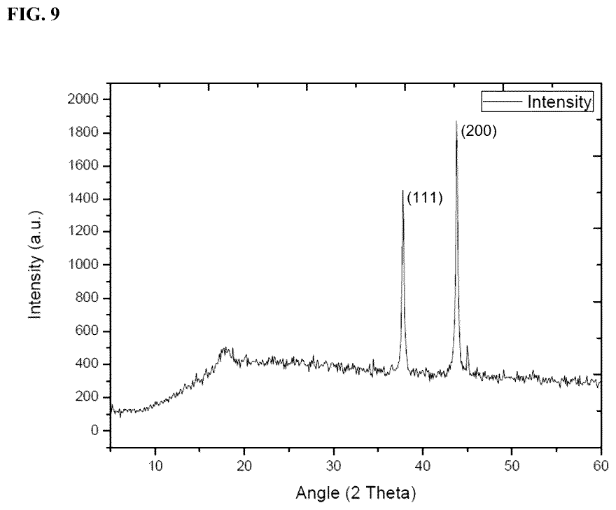

The nickel nanofibers were analyzed by XRD and two sharp peaks of nickel nanoparticles were found (see FIG. 9). The intensity is determined in the range 20°<20<90° with 0.02 degree step size. The 20 values are found to be 37.7922° and 43.8231° respectively. Maximum intensity peak 43.8231° was used to estimate the crystallite size. The peaks at 2 Theta are 37.7922 and 43.8231. The specific diffraction peaks correspond to fcc structure (Nickel, syn, JCPDS card no. 04-0850) (Data based on ICDD/JCPDS PDF Retrievals [Level-1 PDF, Sets 1-51]) (Wei Ni, et al. 2014). It is important to note that only the fcc phase for Ni is present

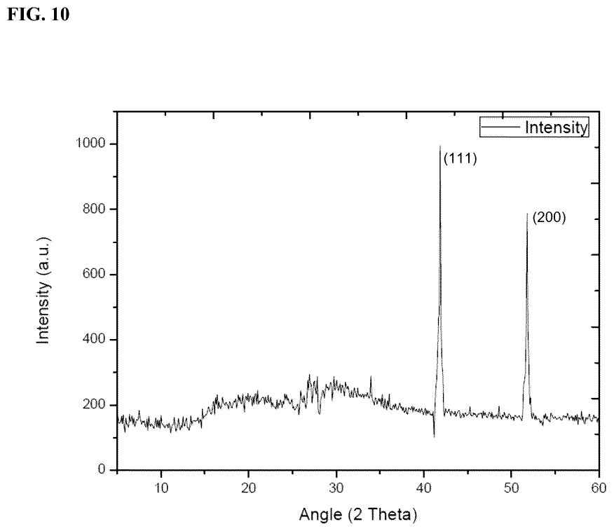

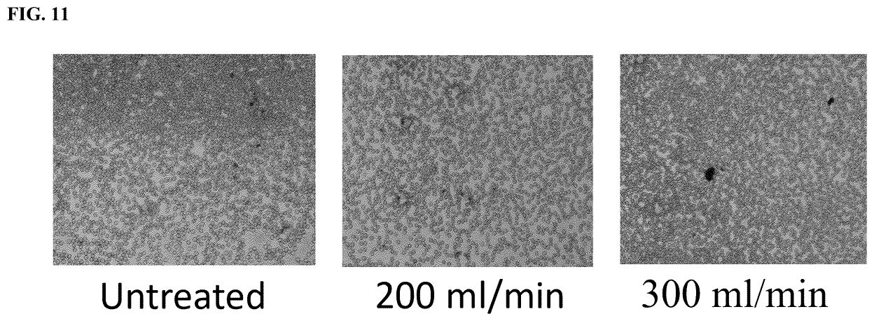

Cobalt nanofibers were analyzed by XRD (FIG. 10). The diffractions obtained at 20=41.87° and 51.79° are relative to the (111) and (200) planes, respectively, indicating the fcc phase of the cobalt spheres (fcc, ICDD/JCPDS No. 15-806) (Qiying Liu et. al., 2015).

### Example 5: Prototype Preparation

### Pre-Prototype Work

A small device (syringe filter) was built using a membrane composed of nanofibers A and B as described above with an effective area of about 0.8 cm² and nanofiber loading about 0.2 mg.

When the contact time of the urea solution with the device was increased to about 40 seconds for filtering 0.6 ml solution, the filtrate did not contain any urea. Additional 0.6 ml urea solution (25 μg/0.6 ml) were passed through the device (and those over the nanofibers) and urea was measured in filtrate each time. By passing 0.1 M NaOH and washing with water, the membrane could be regenerated and it allowed oxidation of urea for at least one cycle as above.

### Prototype Design and Optimization

Two nanofiber compositions were tested and found effective in the removal of urea and ammonia from the blood by hemodialysis method.

Nanofiber-A and Nanofiber B were tested separately in two different cartridges.

Nanofiber A contained 50 μg nickel (0.1 μg/ml) and 150 μg silver (0.3 μg/ml) for the removal of urea; 25 μg of silicon dioxide (0.05 μg/ml) for the removal of ammonia.

Nanofiber B contained 25 μg cobalt (0.05 μg/ml) and 150 μg Silver (0.3 μg/ml) for the removal of urea; 25 μg of tetraphenyl borate (0.05 μg/ml) and 50 μg of silicon dioxide (0.1 μg/ml) for the removal of ammonia.

In a cartridge (housing), nanofibers (A:B ratio 90:10) were added to get the 1.4 m² surface area (exposed to blood for removal of urea.) The quantity of nanoparticles will depend on the size of the cartridge.

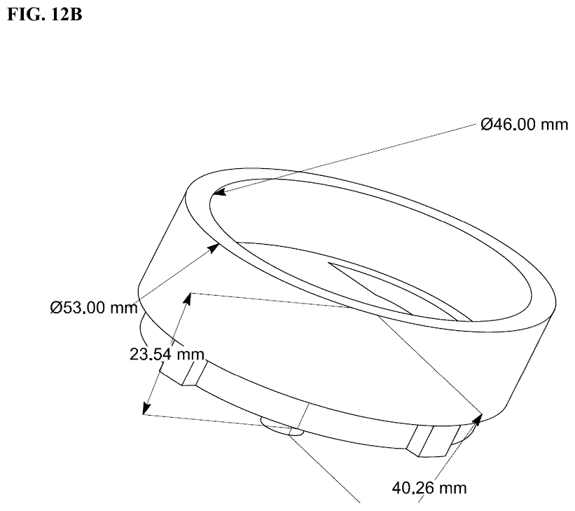

Prototype was designed by third party designer with 3D prototyping as per requirements in Table 9 and EBPG guideline on dialysis strategies, published in 2007 (EBPG guideline on dialysis strategies, Nephrology Dialysis Transplantation, Volume 22, Issue suppl_2, May 2007, Pages ii5-ii21, https://doi.org/10.1093/ndt/gfm022). Acrylonitrile butadiene styrene (ABS) was selected for construction of outer case. The 3D prototype was printed using a 3D printer. The nanofibers were inserted into the prototype manually in upright direction. The cap was placed on the prototype. The dimensions for the prototype are given in Table 9 below and CAD drawings are provided in FIG. 12A-B.

TABLE 9
3D Prototype Details

| Parameter | Details | |
|---|---|---|
| PROTOTYPE HEIGHT | 12.5 | CM |
| PROTOTYPE WIDTH | 4.5 | CM |
| INTERNAL DIAMETER | 4.3 | CM |
| EXTERNAL DIAMETER | 4.5 | CM |
| MEMBRANE SURFACE AREA | 1.4 | m² |

| | |
|---|---|
| POLYMER USED FOR CONJUGATES | Polylactic Acid (PLA) |

### Dialysis Machine

The same diameter tube was used for in and out of the flow through out cartridge. The cartridge was fitted in Nikkiso Dialysis machine and leakage was not observed.

### Flow Rate Optimization

Water (pH 7.0) was used for the initial analysis of the prototype. The water was stored in one container which acted as a reservoir. The following procedure was undertaken: prototype was connected to the hemodialysis machine; single cartridge was used as a prototype; the flow rate was adjusted to 100 ml/min (for 20 min). The prototype was analyzed for leakage, reverse flow and stability. The procedure steps were repeated for 200 ml/min and 300 ml/min. No leakage of the solvents through the dialyzer was observed at 100, 200 and 300 ml/min. No leaching was observed at 100, 200 and 300 ml/min. The incoming and outgoing flow was stable during the extensive testing.

The prototype was used as a dialyzer for urea reduction experiments.

### Urea Reduction and Flow Rate Optimization

Urea solution was prepared (70 mg/dl). The urea solution was stored in one container which acted as a urea reservoir. The flow rate was adjusted to 100 ml/min (for 10 min and 20 min). The urea solution was passed through the dialyzer prototype. Initially, at zero min after first passage of urea solution through the prototype, sample from the outer end was taken for analysis. Samples were taken at 10 and 20 min of exposure to nanofibers through the dialyzer. The urea concentration was estimated. Results are shown in Table 10. The flow rate was adjusted on hemodialysis machine using the control panel of the machine. Control termed “Standard” was a commercial (Nikkiso) (Japan) urea dialysis cartridge.

TABLE 10
Urea degradation by Dialyzer.

| | After zero min | After 10 min | After 20 min |
|---|---|---|---|
| | urea (mg/dl) | urea (mg/dl) | urea (mg/dl) |
| FLOW | 17.5 | 9.23 | 1.4 |
| RATE | | | |
| (100 | | | |
| ml/min) | | | |
| FLOW | 16.98 | 8.99 | 0.91 |
| RATE | | | |
| (200 | | | |
| ml/min) | | | |
| FLOW | 16.44 | 8.74 | 0.69 |
| RATE | | | |
| (300 | | | |
| ml/min) | | | |
| STANDARD | 40.5 | 40.2 | 40.1 |
| Dialyzer | | | |
| Cartridge | | | |

The flow rate was adjusted to 100 ml/min on hemodialysis machine. The urea was estimated by ELISA reader. The first passage of urea solution through the dialyzer was considered as a zero timepoint sample. This sample was taken out in less than one minute. The urea was 17.5 mg/dl compared to untreated urea solution 40 mg/dl. (See top and bottom panel of Table 10). After 10 min of passage through the dialyzer, the urea concentration was 9.23 mg/dl. After 20 min of passage through the dialyzer, the urea concentration was 1.40 mg/dl. (See top and bottom rows of Table 10).

The flow rate was adjusted to 200 ml/min on hemodialysis machine. The urea was 16.98 mg/dl compared to untreated urea solution (40 mg/dl (See second and bottom rows of Table 10—Standard commercial dialyzer cartridge). After 10 min of passage through the dialyzer, the urea concentration was 8.99 mg/dl. After 20 min of passage through the dialyzer, the urea concentration was 0.91 mg/dl.

The flow rate was adjusted to 300 ml/min on hemodialysis machine. The Urea was 16.44 mg/dl compared to untreated urea solution (40 mg/dl) (See third and bottom rows of Table 10—Standard commercial dialyzer cartridge). After 10 min of passage through the dialyzer, the urea concentration was 8.74 mg/dl. After 20 min of passage through the dialyzer, the urea concentration was 0.69 mg/dl.

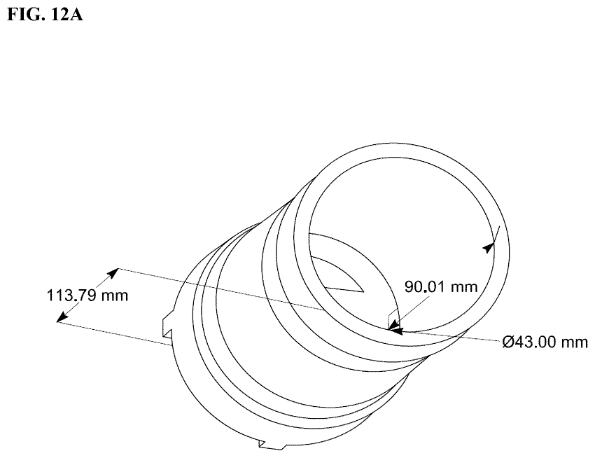

Cells were analyzed after passage through the dialyzer. As shown in FIG. 11, there were no damaged cells found microscopically after 60 min of dialysis at 200 ml/min and 300 ml/min.

Some advantages to a urea removal cartridge as described herein include, but are not limited to, the following: a) treatment time reduces from 3-4 hours to one hour b) The cost of dialysis is reduced, with higher throughput per machine c) Morbidity cost saving to patient as earning loss due to treatment time is eliminated d) The cartridge is designed to achieve nearly 70% reduction of urea in one pass e) Designed to replace current dialyzer cartridges used in hemodialysis f) Cartridge stability (leakproof)—Elemental analysis for possible leaching of nanoparticle ions in blood or water samples g) Limited to no toxicity h) No leakage of the solvents through the dialyzer was observed at different test flow rates. i) No leaching was observed at different test flow rates. j) The incoming and outgoing flow was stable during the extensive testing. k) In-vitro testing of the prototype for urea degradation from urea solutions in 60 min.

### Example 6: Preclinical Study in Rabbit

TABLE 11
Trial and subject details:

| | | |
|---|---|---|
| 1 | Rabbit taken from | PRADO PVT.LTD |
| 2 | Trial conducted at | Venky's Small Animal Clinic and Referral |
| | | Unit, Rahatani, Pune. |
| 3 | Trial conducted on | Oct. 12, 19 |
| 4 | Hemodialysis machine | NIKKISO |
| 5 | Species of rabbit used | New Zealand White rabbit |
| 6 | Age of rabbit | 1.1 yrs (Female) |
| 7 | Body weight of rabbit | 4.8 Kg |

Clinical history of rabbit: On initial presentation, results of the physical examination were unremarkable. The rectal temperature was 37.5° C., the heart rate was 92 beats/min, and the respiratory rate was 18 breaths/min. The rabbit was in good body condition, well-hydrated, and quiet and alert. Rabbit was injected i.p. with cisplatin given in a single dose (6.5 mg/kg). Three days after injection of cisplatin, abnormalities detected included elevated blood urea nitrogen (BUN) (58.7 mg/dl; reference range, 10-33 mg/dl), elevated serum creatinine (4.3 mg/dl; reference range, 0.5-2.2 mg/dl). After that, sample for a complete blood cell count (CBC) and serum biochemical analysis were collected. (Pre-dialysis)

### Surgical Procedure

Anesthesia: The rabbit was sedated using following anesthetic agents via catheter of 22 gauge (G) inserted in the left marginal ear vein. To minimize pain, the marginal region of the ears had been pre-treated with an anesthetic cream one hour before catheterizing the veins. Then, the area was clipped and draped in a sterile manner. After intubation, the rabbits were ventilated with a mixture of air and pure oxygen (4 to 7.5%) at a respiration rate of 40 times per minute.

TABLE 12
Summary of procedure preparation

| Purpose | Anesthetic agent | Dose |
|---|---|---|
| Induction of Anesthesia | Acepromazine | 0.01 mg/kg (I.M.) |
| | Thiopental Na. | 15 mg/kg (I.M.) |
| Maintenance of Anesthesia | Isoflurane | 4% of body weight |
| | | (1^{st} dose) |
| | | (I.V.) |
| | | 2.5% of body weight |
| | | (2^{nd} dose) |
| | | (I.V.) |

Venous Access: The temporary dialysis catheter was placed as follows: The vessel was punctured with a 20 G over-the-needle catheter, using a cut down technique. The guide wire was advanced through the lumen of the 20 G needle, and then the needle was withdrawn. The dilator sheath was placed over the guide wire into the vessel, and the guide wire was withdrawn. The catheter was placed through the dilator sheath into the vessel, the sheath was withdrawn, and the catheter was sutured into place. A lateral radiograph of the thorax was taken to make sure the catheter was placed appropriately and the tip is at the level of the right atrium. The catheter is wrapped in a sterile manner, and is used only for hemodialysis. The catheter was locked with heparin (500 units) to prevent clotting.

Hemodialysis: Priming of hemodialysis machine was done by using normal saline solution (flow rate=300 ml/min). Dialysis catheter tubing was connected from the rabbit to dialyzer. Blood is pulled from one of the 2 ports of the dialysis catheter, and travels through the extracorporeal circuit being pulled (pre-pump) and then pushed (post-pump) by the clockwise circling of the blood pump. Blood then entered into the dialyzer and ran the length of the dialyzer. Filtered blood was then returned to the rabbit via the second port of the dialysis catheter. Initially blood flow rate was kept at 5 ml/min for first 15 min and then it was increased to 15 ml/min throughout the dialysis. Blood pressure was continuously monitored.

Blood Toxicity study: Blood samples were taken at different time points and sent for CBC and biochemistry analysis.

Results are shown below.

TABLE 13
COMPLETE BLOOD COUNT
COMPLETE BLOOD COUNT:

| PRE-DIALYSIS | | | | DURING DIALYSIS | | |
|---|---|---|---|---|---|---|
| | | Ref. | At 0 | At 10 | At 30 | At 60 |
| Test | Unit | value | min | min | min | min |
| Hemoglobin | g/dl | 10.4-17.4 | 15.3 | 15.1 | 14.8 | 14.9 |
| RBC count | 10⁶/μL | 5.46-7.94 | 6.57 | 6.20 | 6.47 | 5.90 |
| Total WBC | Count | 4-10 | 7.61 | 8.32 | 8.57 | 9.73 |
| count | Cells × | | | | | |
| | 10³/cubic | | | | | |
| | mm of | | | | | |
| | blood =/μL | | | | | |

RED CELL ABSOLUTE VALUES

| | | | | | | |
|---|---|---|---|---|---|---|
| Mean | fl | 58.5-66.5 | 62.9 | 63.1 | 63.9 | 63.2 |
| Corp. Volume | | | | | | |
| Mean Corp | pg | 18.7-22.7 | 21.0 | 20.8 | 20.2 | 20.6 |
| HB | | | | | | |

DIFFERENTIAL COUNT

| | | | | | | |
|---|---|---|---|---|---|---|
| Neutrophils | % of | 38-54 | 39 | 38 | 38 | 39 |
| | WBC | | | | | |
| Lymphocytes | % of | 30-70 | 46 | 47 | 46 | 46 |
| | WBC | | | | | |
| Eosinophils | % of | 0-1 | 1 | 1 | 1 | 1 |
| | WBC | | | | | |
| Monocytes | % of | 0-3 | 1 | 1 | 1 | 1 |
| | WBC | | | | | |
| Basophils | % of | 0-1 | 0 | 0 | 0 | 0 |
| | WBC | | | | | |
| Platelets | ×10³/μL | 250-650 | 372 | 370 | 368 | 376 |

PERIPHERAL SMEAR EXAMINATION

| | | | | | | |
|---|---|---|---|---|---|---|
| PLATELETS | — | — | Adequate | Adequate | Adequate | Adequate |
| RBC | — | — | Within | Within | Within | Within |
| Morphology | | | Normal | Normal | Normal | Normal |
| | | | Limit | Limit | Limit | Limit |
| WBC | — | — | Within | Within | Within | Within |
| Morphology | | | Normal | Normal | Normal | Normal |
| | | | Limit | Limit | Limit | Limit |

TABLE 14
Results: BIOCHEMISTRY

| PRE-DIALYSIS | | | | DURING DIALYSIS | | |
|---|---|---|---|---|---|---|
| | | Reference | At 0 | At 10 | At 30 | At 60 |
| Test | Unit | value | min | min | min | min |
| Blood Urea | mg/dl | 10-33 | 58.7 | 41.1 | 33.5 | 27.3 |
| Nitrogen | | | | | | |
| Serum | mg/dl | 0.5-2.2 | 4.3 | 4.3 | 4.2 | 4.2 |
| Creatinine | | | | | | |
| Serum | mmol/lit | 140-160 | 153 | 155 | 155 | 159 |
| Sodium | | | | | | |
| Serum | mmol/lit | 4.3-5.8 | 5.1 | 4.9 | 4.3 | 4.8 |
| Potassium | | | | | | |
| Serum | mmol/lit | 90-110 | 94 | 96 | 96 | 95 |
| Chloride | | | | | | |
| Serum | mg/dl | 8.0-15.5 | 11.8 | 12.2 | 12.8 | 12.1 |
| Calcium | | | | | | |
| Blood | mg/dl | 80-150 | 110 | 108 | 108 | 111 |
| Glucose | | | | | | |
| Vitamin B12 | ng/ml | — | 156 | 153 | 157 | 156 |

Estimation of urea from blood proved that there was reduction in urea level from 58.7 mg/dl to 27.3 mg/dl within 60 min. This demonstrated that the nanofiber composition herein was capable of reducing urea up to 31.4 mg/dl.

### Example 7: Preclinical Study in Canine

TABLE 15
Trial and subject details:

| 1 | Trial conducted at | Venky's Small Animal Clinic and | |
|---|---|---|---|
| | | Referral Unit, Rahatani, Pune, India | |
| 2 | Trial conducted on | 27 Nov. 2019 | |
| 3 | Hemodialysis machine | NIKKISO | |
| 4 | Species of dog used | German Boxer | |
| 5 | Sex of dog | Female | |
| 6 | Age of dog | 8 | yrs |
| 7 | Body weight of dog | 22 | kg |

Clinical history of Dog: On initial presentation, results of the physical examination were unremarkable. The rectal temperature was 37.5° C., the heart rate was 95 beats/min, and the respiratory rate was 16 breaths/min. The dog was in good body condition, well-hydrated, and quiet and alert. Auscultation of the thorax revealed normal lung sounds in all fields. Results of abdominal palpation were normal. Abnormalities associated with canine kidney problems were detected including elevated blood urea nitrogen (BUN) (64 mg/dl; reference range, 6-25 mg/dl), elevated serum creatinine (2.65 mg/dl; reference range, 0.5-1.6 mg/dl). The mean arterial blood pressure was 100 mmHg. A diagnosis of acute renal failure (ARF) was suspected, based on the short duration of signs including significant azotemia. After physical examination, samples for a complete blood cell count (CBC) and serum biochemical analysis were collected.

### Surgical Procedure

Anesthesia: The dog was sedated using the anesthetic agents described in the table below. Then, the area was clipped and draped in a sterile manner.

TABLE 16
Summary of procedure preparation

| Purpose | Anaesthetic agent | Dose |
|---|---|---|
| Induction of Anesthesia | Acepromazine | 0.01 mg/kg (I.M.) |
| | Thiopental Na. | 15 mg/kg (I.M.) |
| Maintenance of | Isoflurane | 4% of body weight |
| Anesthesia | | (1^{st} dose) |
| | | (I.V.) |
| | | 2.5% of body weight |
| | | (2^{nd} dose) |
| | | (I.V.) |

Venous Access: The temporary dialysis catheter was placed as follows. The vessel was punctured with a 16 G over-the-needle catheter, using a cut down technique. The guide wire was advanced through the lumen of the 16 G needle, and then the needle withdrawn. The dilator sheath was placed over the guide wire into the vessel, and the guide wire was withdrawn. The catheter was placed through the dilator sheath into the vessel, the sheath was withdrawn, and the catheter was sutured into place. A lateral radiograph of the thorax was taken to make sure the catheter was placed appropriately and the tip was at the level of the right atrium. The catheter was wrapped in a sterile manner, and was used only for hemodialysis. The catheter was locked with heparin (500 units) to prevent clotting.

Hemodialysis: Priming of hemodialysis machine was done by using normal saline solution (flow rate=300 ml/min). Dialysis catheter tubing was connected from the dog and attached to the dialyzer. Blood was pulled from one of the 2 ports of the dialysis catheter, and traveled through the extracorporeal circuit being pulled (pre-pump) and then pushed (post-pump) by the clockwise circling of the blood pump. Blood then entered into the dialyzer and ran the length of the dialyzer. Filtered blood was then returned to the dog via the second port of the dialysis catheter. Initially blood flow rate was kept at 5 ml/min for first 15 min and then it was increased to 20 ml/min throughout the dialysis. Blood pressure was continuously monitored.

Blood Toxicity study. Blood samples were taken at different time points and sent for analysis.

Results are shown below.

TABLE 17
COMPLETE BLOOD COUNT:

| PRE-DIALYSIS | | | | DURING DIALYSIS | | |
|---|---|---|---|---|---|---|
| | | Reference | At 0 | At 10 | At 30 | At 60 |
| Test | unit | value | min | min | min | min |
| HAEMOGLOBIN | g/dl | 12.1-20.3 | 11.4 | 11.1 | 11.3 | 11.2 |
| RBC COUNT | cmm | 4.8-9.3 | 4.12 | 4.82 | 4.14 | 4.10 |
| TOTAL WBC | Cells × | 4000-15500 | 17000 | 17000 | 17600 | 17200 |
| COUNT | 10³/cubic | | | | | |
| | mm of | | | | | |
| | blood =/μL | | | | | |

RED CELL ABSOLUTE VALUES

| | | | | | | |
|---|---|---|---|---|---|---|
| MEAN | fl | 58-79 | 64 | 61 | 61 | 65 |
| CORP. VOLUME | | | | | | |
| MEAN CORP HB | pg | 19-28 | 27 | 25 | 28 | 27 |

DIFFERENTIAL COUNT

| | | | | | | |
|---|---|---|---|---|---|---|
| NEUTROPHILS | % of WBC | 60-77 | 82 | 80 | 81 | 81 |
| LYMPHOCYTES | % of | 12-30 | 15 | 15 | 14 | 15 |
| | WBC | | | | | |
| EOSINOPHILS | % of | 2-10 | 1 | 2 | 2 | 2 |
| | WBC | | | | | |
| MONOCYTES | % of | 3-10 | 2 | 3 | 3 | 2 |
| | WBC | | | | | |
| BASOPHILS | % of | 0-1 | 0 | 0 | 0 | 0 |
| | WBC | | | | | |

PERIPHERAL SMEAR EXAMINATION

| | | | | | | |
|---|---|---|---|---|---|---|
| PLATELETS | — | — | Adequate | Adequate | Adequate | Adequate |
| RBC | — | — | Within | Within | Within | Within |
| MORPHOLOGY | | | Normal | Normal | Normal | Normal |
| | | | Limit | Limit | Limit | Limit |
| WBC | — | — | Within | Within | Within | Within |
| MORPHOLOGY | | | Normal | Normal | Normal | Normal |
| | | | Limit | Limit | Limit | Limit |

TABLE 18
BIOCHEMISTRY

| PRE-DIALYSIS | | | | DURING DIALYSIS | | |
|---|---|---|---|---|---|---|
| | | Reference | At 0 | At 10 | At 30 | At 60 |
| Test | unit | value | min | min | min | min |
| BLOOD UREA | mg/dl | 6-25 | 64 | 38 | 20 | 17 |
| NITROGEN | | | | | | |
| SERUM | mg/dl | 0.5-1.6 | 2.65 | 2.60 | 2.59 | 2.60 |
| CREATININE | | | | | | |
| Serum. Sodium | mmol/lit | 139-154 | 160 | 158 | 161 | 159 |
| Serum | mmol/lit | 3.6-5.5 | 6.80 | 6.30 | 6.60 | 6.50 |
| Potassium | | | | | | |
| Serum Chloride | mmol/lit | 102-120 | 118.0 | 114 | 115 | 114 |
| Serum Calcium | mg/dl | 8.9-11.4 | 10.90 | 10.20 | 10.20 | 10.40 |
| Vitamin B12 | ng/ml | 200-900 | 429 | 409 | 402 | 398 |

1. A nanofiber composition comprising a polymer and nanoparticles comprising one or more of nickel, cobalt, silver, and tetraphenylborate.

2. The composition of claim 1, wherein the polymer comprises one or more of silicon dioxide, polyurethane prepolymer (PUP), polylactic acid (PLA), polycarbonate, polyvinyl alcohol (PVA), polyacrylic acid (PAA), polyethylene glycol (PEG), and polyvinyl pyrrolidone (PVP).

3. The nanofiber composition of claim 1, wherein the nanoparticles comprise nickel.

4. The nanofiber composition of claim 1, wherein the nanoparticles comprise cobalt.

5. The nanofiber composition of claim 1, wherein the nanoparticles comprise silver.

6. The nanofiber composition of claim 1, wherein the nanoparticles comprise tetraphenylborate.

7. The composition of claim 1, wherein the nanofiber composition comprises nickel nanoparticles and cobalt nanoparticles.

8. The composition of claim 1, wherein the nanofiber composition comprises nickel nanoparticles and silver nanoparticles.

9. The composition of claim 1, wherein the nanofiber composition comprises cobalt nanoparticles and silver nanoparticles.

10. The composition of claim 1, wherein the nanofiber composition comprises nickel nanoparticles, cobalt nanoparticles, silver nanoparticles, and tetraphenylborate nanoparticles.

11. The composition of claim 1, wherein the polymer comprises silicone dioxide and the nanoparticles comprise nickel and cobalt.

12. The composition of claim 1, wherein the polymer comprises silicone dioxide and the nanoparticles comprise silver and cobalt.

13. The composition of claim 11, wherein the polymer further comprises polyvinyl pyrrolidone (PVP).

14. The composition of claim 11, further comprising tetraphenylborate nanoparticles.

15.-16. (canceled)

17. A cartridge comprising one or more membranes, wherein each membrane comprises a nanofiber composition, wherein the nanofiber composition comprises a polymer and one or more nanoparticles comprising nickel, cobalt, silver, and/or tetraphenylborate.

18.-28. (canceled)

29. A device comprising:
a. a filtration chamber configured to receive blood comprising urea; and
b. one or more membranes disposed within said filtration chamber, wherein each membrane comprises nanofibers comprising a polymer and nanoparticles comprising one or more of nickel, cobalt, silver, and tetraphenylborate, and wherein the nanofibers are capable of binding urea, converting urea to ammonia, and subsequently binding ammonia.

30-31. (canceled)

32. A method of reducing the concentration of urea from blood, comprising:
a. providing blood comprising urea to a device comprising a cartridge, wherein the cartridge comprises one or more membranes, wherein each membrane comprises a nanofiber composition of claim 1;
b. contacting the blood with the membrane for a sufficient amount of time to allow binding of urea and conversion of urea to ammonia; and
c. pumping the blood through the cartridge at a sufficient pressure to allow binding of ammonia to the membrane, thereby reducing the concentration of urea in the blood.

33. A method of treating a subject with a disease condition characterized by elevated blood urea concentration, the method comprising:
a. obtaining a sample of the subject's blood;
b. pumping the sample through a nanofiber composition of claim 1, for a time period sufficient to allow the nanofiber composition to bind urea, convert urea to ammonia, and subsequently bind ammonia, thereby creating a filtered blood sample; and
c. returning the filtered blood sample to the subject, thereby treating the subject.

34. A method of treating a mammal for elevated blood urea concentration comprising:
a. providing blood comprising urea to a device comprising one or more membranes, wherein each membrane comprises a nanofiber composition of claim 1, and wherein the nanofibers are capable of binding urea, converting urea to ammonia, and subsequently binding ammonia,
b. contacting the blood with the membrane for a sufficient amount of time to allow binding of urea and conversion to ammonia; and
c. pumping the blood through the cartridge at a sufficient pressure and flow rate to allow binding of ammonia to the nanofibers, thereby reducing the concentration of urea in the blood.

35. A method of treating a subject with a disease condition characterized by elevated urea concentration in blood, the method comprising:
a. administering ex vivo hemofiltration to said subject wherein blood is removed from the subject and filtered through a device comprising a filtration chamber (1) configured to receive blood comprising elevated urea concentration, and (2) composed of one or more membranes, wherein each membrane comprises a nanofiber composition of claim 1;
b. incubating the blood or plasma with the one or more membranes for a sufficient time to allow binding of urea to the nanofiber, conversion of urea to ammonia, and subsequent binding of ammonia to the nanofibers;
c. pumping the blood through the device at a pressure and flow rate sufficient to filter the blood and produced an amount of filtered blood; and
d. returning filtered blood to said subject, wherein the filtered blood is reduced in urea concentration by at least about 50%.

36-38. (canceled)
